(19) United States
(12) Patent Application Publication
(10) Pub. No.: US 2026/0286363 A1
(43) Pub. Date: Sep. 24, 2026

(54) COMPOSITIONS AND METHODS FOR DIAGNOSING, TREATING, AND PREVENTING LYSOSOMAL STORAGE DISEASES

(71) Applicant: Wylder Nation Foundation, G5-574 Scottsdale, AZ (US)

(72) Inventors: Maria Dolores LEDESMA, Madrid (ES); Edward H. SCHUCHMAN, New York, NY (US); Angel GAUDIOSO, Madrid (ES)

(21) Appl. No.: 18/997,973

(22) PCT Filed: Jul. 27, 2023
(86) PCT No.: PCT/US2023/071095

§ 371 Date: Jan. 23, 2025

Related U.S. Application Data
(60) Provisional application No. 63/369,721, filed on Jul. 28, 2022.

(51) Int. Cl. C12N 15/113; A61K 38/46; A61P 43/00; C12N 15/11; C12N 15/86; G01N 33/92

(52) U.S. Cl. CPC C12N 15/1137; A61K 38/465; A61P 43/00; C12N 15/11; C12N 15/86; C12Y 203/01024; C12Y 301/04012; G01N 33/92; C12N 2310/20; C12N 2310/531; C12N 2320/31; C12N 2750/14143; G01N 2405/08; G01N 2800/38; G01N 2800/50

(57) ABSTRACT

The present disclosure provides compositions and methods of identifying, making, and using the same for the prevention and treatment of lysosomal storage diseases. Provided are also compositions and methods of diagnosing lysosomal storage diseases.

### CROSS-REFERENCE TO RELATED APPLICATIONS

This application claims priority to U.S. Application No. 63/369,721 filed on Jul. 28, 2022, which is incorporated by reference herein in its entirety for all purposes.

### REFERENCE TO AN ELECTRONIC SEQUENCE LISTING

The contents of sequence the electronic listing (WYNF_003_00US_SeqList_ST26.xml; Size: 22,171 bytes; and Date of Creation: Jul. 27, 2022) is herein incorporated by reference in its entirety.

### FIELD OF DISCLOSURE

The present disclosure generally relates to compositions and methods useful in the prevention and treatment of lysosomal storage diseases. The present disclosure also relates to diagnostic reagents and methods of using the same to identify lysosomal storage diseases in subjects.

### BACKGROUND

Acid sphingomyelinase deficiency (ASMD) is a lysosomal storage disorder caused by mutations in the acid sphingomyelinase (ASM) gene. While intravenous infusion of recombinant ASM seems promising to treat the peripheral disease, the neurological condition remains unaddressed.

Provided herein are compositions and methods of diagnosing, treating, and preventing lysosomal storage diseases such as ASMD.

### BRIEF SUMMARY

Provided herein are methods of determining an increased risk of a lysosomal storage disease in a subject, the methods comprising determining an increased level of sphingomyelin in the subject as compared to a baseline level of the subject or an otherwise comparable healthy subject. In aspects, the increased level of sphingomyelin is detected in a blood sample of the subject. In aspects, the increased level of sphingomyelin is detected in a sample from the central nervous system of the subject. In aspects, the sample from the central nervous system comprises cerebrospinal fluid. In aspects, the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof. In aspects, the sphingomyelin is sphingomyelin 16:0. In aspects, the lysosomal storage disease comprises acid sphingomyelinase deficiency. In aspects, the subject is pediatric. In aspects, the subject is an infant. In aspects, the infant is a newborn infant. In aspects, the determining is completed within about 24 hours, 48 hours, or 72 hours of birth.

Provided are methods of preventing or treating a lysosomal storage disease, the methods comprising administering an agent in an amount effective to reduce a level of sphingomyelin in the brain of a subject in need thereof, wherein the agent targets a metabolic enzyme that comprises a ceramide synthase. In aspects, the ceramide synthase is selected from the group consisting of CerS4, CerS5, and CerS6. In aspects, the ceramide synthase is CerS5. In aspects, the metabolic enzyme consists of the ceramide synthase. In aspects, the agent inhibits the metabolic enzyme, silences an RNA encoding the metabolic enzyme, or genomically disrupts a nucleic acid encoding the metabolic enzyme. In aspects, the agent silences the RNA encoding the metabolic enzyme. In aspects, the agent comprises an adeno-associated virus (AAV) expressing a shRNA that silences the RNA encoding the metabolic enzyme. In aspects, the shRNA comprises a sense sequence, loop sequence, and antisense sequence comprising at least about 80% identity to SEQ ID NO: 23, TCAAGAG, and SEQ ID NO: 24, respectively. In aspects, the shRNA comprises the sense sequence, loop sequence, and antisense sequence of SEQ ID NO: 23, TCAAGAG, and SEQ ID NO: 24, respectively. In aspects, the AAV is of a serotype selected from the group consisting of: AAV2, AAV5, AAV6, AAV8, AAV9, a portion thereof, a fusion product thereof, and any combination thereof. In aspects, the AVV is of serotype AAV9. In aspects, the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof. In aspects, the sphingomyelin is sphingomyelin 16:0. In aspects, the lysosomal storage disease comprises acid sphingomyelinase deficiency. In aspects, the level is reduced by at least about 1-fold, 5-fold, 20-fold, or 50-fold as compared to an otherwise comparable method lacking the administering. In aspects, the subject is a pediatric subject. In aspects, the subject is an adult. In aspects, the subject is concurrently administered recombinant acid sphingomyelinase. In aspects, the subject was previously administered recombinant acid sphingomyelinase.

Provided are compositions comprising: a) an inhibitor of a ceramide synthase; b) a short hairpin RNA (shRNA) targeting a ceramide synthase; c) a guide RNA that targets a nucleic acid sequence encoding a ceramide synthase; or d) a guide RNA that targets a ribonucleic acid sequence encoding acid sphingomyelinase. In aspects, compositions comprise the shRNA. In aspects, the shRNA comprises a sense sequence, loop sequence, and antisense sequence comprising at least about 80% identity to SEQ ID NO: 23, TCAAGAG, and SEQ ID NO: 24, respectively. In aspects, the shRNA comprises the sense sequence, loop sequence, and antisense sequence of SEQ ID NO: 23, TCAAGAG, and SEQ ID NO: 24, respectively.

Provided are AAV particles comprising a sequence encoding a short hairpin RNA (shRNA) targeting a ceramide synthase. In aspects, particles are of a serotype selected from the group comprising: AAV2, AAV5, AAV6, AAV8, AAV9, a portion thereof, a fusion product thereof, and any combination thereof. In aspect, particles are of serotype AAV9. In aspects, the shRNA comprises a sense sequence, loop sequence, and antisense sequence comprising at least about 80% identity to SEQ ID NO: 23, TCAAGAG, and SEQ ID NO: 24, respectively. In aspects, the shRNA comprises the sense sequence, loop sequence, and antisense sequence of SEQ ID NO: 23, TCAAGAG, and SEQ ID NO: 24, respectively. In aspects, a ceramide synthase is selected from the group consisting of: CerS4, CerS5, and CerS6. In aspects, a ceramide synthase is CerS5.

Provided are a plurality of AAV particles. In aspects, the plurality is in unit dose form.

Provided are pharmaceutical compositions comprising: a) compositions of the disclosure, AAV particles of the disclosure; and b) a pharmaceutically acceptable excipient, carrier, or diluent.

Provided are methods of treating or preventing a disease or a condition in a subject in need thereof, the methods comprising: administering to the subject the pharmaceutical composition of the disclosure thereby treating or preventing the disease or the condition. In aspects, the administering is intrathecally, intraocularly, intravitreally, retinally, intravenously, intramuscularly, intraventricularly, intracerebrally, intracerebrally, intracerebroventricularly, intraperenchymally, subcutaneously, or a combination thereof. In aspects, the administering is intrathecal. In aspects, the disease or condition is a lysosomal storage disease. In aspects, the lysosomal storage disease comprises acid sphingomyelinase deficiency. In aspects, after the administering, the subject comprises: a) at least a 1-fold reduced formation of sphingomyelin as compared to an otherwise comparable subject lacking the administering, as measured by: a brain scan, a blood test, or both; or b) at least a 1-fold increase in acid sphingomyelinase, as compared to an otherwise comparable subject lacking the administering, as determined a brain scan, a blood test, or both.

Provided are AAV particles comprising a sequence encoding a short hairpin RNA (shRNA) targeting a ceramide synthase, wherein the shRNA targets a sequence that is bound by or complementary to at least one of SEQ ID NO: 23 or SEQ ID NO: 25. In aspects, the AAV particles are of serotype AAV9. In aspects, the ceramide synthase is CerS5.

Provided are methods of preventing or treating acid sphingomyelinase deficiency comprising administering AAV particles of the disclosure to a subject in need thereof, thereby preventing or treating the acid sphingomyelinase deficiency. In aspects, the administering is effective in reducing an amount of sphingomyelin 16:0 in the subject in need thereof as compared to an otherwise comparable subject lacking the administering.

Provided are methods of determining whether an agent is neuromodulatory, the methods comprising determining a blood plasma concentration of sphingomyelin in a subject treated with an agent, wherein a decrease in the blood plasma concentration as compared to a baseline level is indicative of the efficacy of the compound as a neuromodulatory agent. In aspects, wherein when the decrease is detected, the subject continues treatment with the agent. In aspects, the subject has a lysosomal storage disease. In aspects, the lysosomal storage disease comprises acid sphingomyelinase deficiency. In aspects, the subject is pediatric. In aspects, the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof. In aspects, the sphingomyelin is sphingomyelin 16:0.

Provided are methods of selecting a biomarker, the methods comprising: administering a composition comprising ASM to a subject; and detecting a level of a lipid in the subject after the administering, wherein when the level of the lipid decreases in a liver of the subject but not a brain or plasma, the lipid is selected. In aspects, the lipid comprises a phosphocholine. In aspects, the phosphocholine comprises m/z805.

Provided are compositions comprising m/z805.

These and other embodiments are described below.

### BRIEF DESCRIPTION OF THE DRAWINGS

The accompanying figures, which are incorporated herein and form a part of the specification, illustrate some, but not the only or exclusive, example aspects and/or features. It is intended that the aspects and figures disclosed herein are to be considered illustrative rather than limiting.

FIG. 1A-FIG. 1D show variable relative increase of SM species in the ASMko brain and cultured neurons. FIG. 1A shows mean±SEM of total SM levels expressed as nmol/mg protein in extracts from the cerebral cortex and cerebellum of wt and ASMko mice at 4.5 months of age (n=5; ****p<0.0001). FIG. 1B shows mean±SEM levels of the indicated SM species expressed as nmol/mg protein in extracts from the cerebral cortex and cerebellum of wt and ASMko mice at 4.5 months of age (n=5; *p<0.05; **p<0.01; ***p<0.001; ****p<0.0001). FIG. 1C shows mean percentages of the indicated SM species with respect to total SM in extracts from the cerebral cortex and cerebellum of wt and ASMko mice at 4.5 months of age (n=5). FIG. 1D shows mean±SEM levels of total SM expressed as nmol/mg protein (left) or mean percentages of SM species with respect to total SM in extracts from cultured cortical neurons from wt and ASMko mice (n=3 independent cultures; **p<0.01).

FIG. 2A-FIG. 2F show the differing impact of SM species when added to cultured wt neurons. FIG. 2A depicts mean±SEM of total SM levels in cultured wt neurons incubated with the indicated SM species expressed as nmol/mg protein (n=3 independent cultures; **p<0.01; ***p<0.001; ****p<0.0001). FIG. 2B shows mean percentages of SM species with respect to total SM in cultured wt neurons incubated with the SM species indicated in the X axis or with vehicle. FIG. 2C shows images of Lysotracker staining in cultured wt neurons treated with vehicle or the indicated SM species. Insets in the upper images are magnified in the lower panel. Bar=20 μm. Bar (high magnification)=5 μm. FIG. 2D shows immunofluorescence against the lysosomal marker Lamp1 in non-permeabilized cultured wt neurons treated with vehicle or the indicated SM species. Graph shows mean±SEM Lamp1 surface staining expressed as fold-increase with respect to vehicle treated cultures (n=3 independent cultures; *p<0.05; **p<0.01). Bar=20 μm. FIG. 2E shows DHR staining in cultured wt neurons treated with vehicle or the indicated SM species. FIG. 2F shows mean #SEM DHR intensity (directly proportional to ROS levels) expressed as fold-increase with respect to vehicle treated cultures (n=4 independent cultures; *p<0.05; **p<0.01). Bar=20 μm. The right panel shows mean±SEM cellular viability measured by MTT in cultured wt neurons in treated with vehicle or the indicated SM species and expressed as percentage of living cells with respect to vehicle treated cultures (n=3 independent cultures; *p<0.05).

FIG. 3A-FIG. 3G show that SM16:0 targets lysosomes in cultured neurons. FIG. 3A shows merged low magnification image (left) and single-channel magnified insets (right) of cultured wt neurons incubated with the fluorescent analogue pSM 16:0 co-stained by immunofluorescence against the plasma membrane marker Thy 1 (n=3 independent cultures). Bar=20 μm. Bar (high magnification)=5 μm. FIG. 3B shows merged low magnification image (left) and single-channel magnified insets (right) of cultured wt neurons incubated with the fluorescent analogue pSM16:0 co-stained by immunofluorescence against the postsynaptic density marker PSD95. Bar=20 μm. Bar (high magnification)=5 μm. FIG. 3C shows merged low magnification image (left) and single-channel magnified insets (right) of cultured wt neurons incubated with the fluorescent analogue pSM16:0 co-stained by immunofluorescence against the mitochondrial marker TOM20. Bar=20 μm. Bar (high magnification)=5 μm. FIG. 3D shows merged low magnification image (left) and single-channel magnified insets (right) of cultured wt neurons incubated with the fluorescent analogue pSM16:0 co-stained by immunofluorescence against the early endosomal marker EEA1. Bar=20 μm. Bar (high magnification)=5 μm. FIG. 3E shows merged low magnification image (left) and single-channel magnified insets (right) of cultured wt neurons incubated with the fluorescent analogue pSM16:0 co-stained by immunofluorescence against the late endosome-lysosomal marker LAMP1. Bar=20 μm. Bar (high magnification)=5 μm. FIG. 3F shows merged low magnification image (left) and single-channel magnified insets (right) of cultured wt neurons incubated with the fluorescent analogue pSM16:0 co-stained by immunofluorescence against the Golgi marker GM130. Bar=20 μm. Bar (high magnification)=5 μm. FIG. 3G shows mean±SEM Mander's coefficient of colocalization between pSM16:0 and the indicated subcellular markers Thy1, PSD95, TOM20, EEA1, LAMP1 or GM130 (n=3 independent cultures).

FIG. 4A-FIG. 4C show altered gene expression of SM metabolic enzymes in the brain of ASMko mice. FIG. 4A depicts mean±SEM expression levels of CerS1-6 genes encoding for CerS1-6 measured by qPCR in cortical or cerebellar extracts of wt and ASMko mice. Data are expressed in arbitrary units normalized to the values obtained in wt mice that were considered 1 (n=5; *p<0.05). FIG. 4B shows mean±SEM expression levels of the Sgms1-2 genes encoding for SMS1-2, measured by qPCR in cortical and cerebellar extracts of wt and ASMko mice. Data are expressed in arbitrary units normalized to the values obtained in wt mice that were considered 1 (n=5; *p<0.05). FIG. 4C depicts mean±SEM expression levels of the Smpd2-4 genes encoding for Smases2-4, measured by qPCR in cortical and cerebellar extracts of wt and ASMko mice. Data are expressed in arbitrary units normalized to the values obtained in wt mice that were considered 1. (n=5; *p<0.05).

FIG. 5A-FIG. 5F show the beneficial effects of CerS5 genetic silencing in ASMko cultured neurons. FIG. 5A is a western Blot against CerS5 and against β-actin as loading control in cerebellar extracts of wt and ASMko mice. Graph shows mean±SEM CerS5 protein expression in the ASMko cerebellar extracts normalized to β-actin and expressed as fold-increase with respect to wt extracts (n=3; *p<0.05). FIG. 5B is a western Blot against CerS5 and against β-actin as loading control in cultured neurons from ASMko mice infected with adenovirus containing either shRNA-CerS5 or shRNA-scramble. Graph shows mean±SEM CerS5 protein expression normalized to β-actin and expressed as fold-increase with respect to shRNA-scramble infected cultures (n=3; *p<0.05). FIG. 5C depicts mean±SEM levels of total SM expressed as nmol/mg protein (left) or mean percentages of SM species with respect to total SM in cultured ASMko neurons infected with adenovirus containing either shRNA-CerS5 or shRNA scramble (n=4 independent cultures). FIG. 5D shows immunofluorescence against the lysosomal marker LAMP1 in cultured ASMko neurons infected with adenovirus containing either shRNA-CerS5 or shRNA-scramble. Graph shows mean±SEM lysosomal area expressed as fold-change with respect to the shRNA-scramble infected cultures (n=4 independent cultures: *p<0.05) Bar=20 μm. Bar (high magnification)=5 μm. FIG. 5E are representative images of Lysotracker staining in cultured ASMko neurons infected with adenovirus containing either shRNA-CerS5 or shRNA-scramble. Bar=20 μm. Bar (high magnification)=5 μm. FIG. 5F are representative images of DHR staining in cultured ASMko neurons infected with adenovirus containing either shRNA-CerS5 or shRNA-scramble. Graph shows mean±SEM DHR intensity (directly proportional to ROS levels) expressed as fold-change with respect to shRNA-scramble infected cultures (n=4 independent cultures: **p<0.01) Bar=20 μm.

FIG. 6A-FIG. 6F show the beneficial effects of CerS5 genetic silencing in the cerebellum of ASMko mice. FIG. 6A shows a western Blot against CerS5 and against β-actin as loading control in cerebellar extracts of wt and ASMko mice injected with AAV9-shRNA-CerS5 or AAV9-shRNA scramble. Graph shows mean±SEM CerS5 protein expression in the cerebellar extracts normalized to β-actin and expressed as fold-change with respect to the shRNA scramble injected samples (n=3). FIG. 6B depicts mean±SEM levels of total SM expressed as nmol/mg protein (left) or mean percentages of SM species with respect to total SM in cerebellar extracts (right) of wt and ASMko mice injected with AAV9-shRNA-CerS5 or AAV9-shRNA-scramble (n=4; ***p<0.001). FIG. 6C shows immunofluorescence against the Purkinje cell marker Calbindin in the cerebellum of wt and ASMko mice injected with AAV9-shRNA-CerS5 or AAV9-shRNA-scramble. Graph shows mean±SEM number of Purkinje cells (n=4; *p<0.05; ****p<0.0001) Bar=500 μm. FIG. 6D shows immunofluorescence against the lysosomal marker Lamp1 in Purkinje cells of the cerebellum of wt and ASMko mice injected with AAV9-shRNA-CerS5 or AAV9-shRNA-scramble. Graph shows mean±SEM lysosomal area (n=4; **p<0.01; ****p<0.0001). Bar-20 μm. FIG. 6E shows immunofluorescence against the microglia marker Iba1 in the cerebellum of wt and ASMko mice injected with AAV9-shRNA-CerS5 or AAV9-shRNA-scramble. Graphs show mean±SEM microglia number or area (n=4; *p<0.05; ****p<0.0001). Bar=100 μm. FIG. 6F shows immunofluorescence against the astrocytic marker GFAP in the cerebellum of wt and ASMko mice injected with AAV9-shRNA-CerS5 or AAV9-shRNA-scramble. Graphs show mean±SEM intensity associated to GFAP per area unit (n=4; *p<0.05). Bar=100 μm.

FIG. 7A-FIG. 7F show that increased SM 16:0 levels in plasma correlate with brain pathology in ASMko mice. FIG. 7A is a heatmap showing the relative increments in the SM species levels in liver and brain of ASMko compared to wt mice (n=5). FIG. 7B depicts the linear regression of total SM levels in plasma (x-axis) and brain or liver (y-axis) of wt and ASMko mice at 4.5 months of age. The correlation coefficient (r) and the p value of each correlation are indicated (n=7). *p<0.05. FIG. 7C shows the linear regression of SM16:0 levels in plasma (x-axis) and brain or liver (y-axis) of wt and ASMko mice at 4.5 months of age. The correlation coefficient (r) and the p value of each correlation are indicated (n=7). *p<0.05. FIG. 7D depicts the linear regression of SM18:0 levels in plasma (x-axis) and brain or liver (y-axis) of wt and ASMko mice at 4.5 months of age. The correlation coefficient (r) and the p value of each correlation are indicated (n=7). *p<0.05. FIG. 7E shows the linear regression of SM24:1 levels in plasma (x-axis) and brain or liver (y-axis) of wt and ASMko mice at 4.5 months of age. The correlation coefficient (r) and the p value of each correlation are indicated (n=7). *p<0.05. FIG. 7F shows the linear regression of SM16:0 levels in plasma (x-axis) and brain (y-axis) of wt and ASMko mice at 2, 4.5 and 6 months of age. The correlation coefficient (r) and the p value of each correlation are indicated (n=18). *p<0.05.

FIG. 8 shows that SM16:0 induces cell death in the presence of sphingomyelinase inhibitors. The graph shows mean±SEM cellular viability measured by MTT in cultured wt neurons treated with vehicle or the indicated SM species in the presence of Desipramine (which inhibits the ASmase) or GW4569 (which inhibits the NSmase). Data are expressed as percentage of living cells with respect to vehicle treated cultures (n=3 independent cultures; ***p<0.001).

FIG. 9A-FIG. 9B show that CerS5 silencing reduces dihydroceramide 16:0 levels. FIG. 9A shows mean±SEM dihydroceramide levels expressed as nmol/mg protein in cultured ASMko neurons infected with adenoviral vectors containing either shRNA-CerS5 or shRNA scramble (n=4 independent cultures). FIG. 9B shows mean±SEM dihydroceramide levels expressed as nmol/mg protein in cerebellar extracts of ASMko mice injected with AAV9-shRNA-CerS5 or AAV9-shRNA-scramble (n=4; *p<0.05).

FIG. 10 shows that SM16:0 levels in CSF correlate with those in brain of ASMko mice. Depicted is the linear regression of SM16:0 levels in CSF (x-axis) and brain or liver (y-axis) of wt and ASMko mice at 4.5 months of age. The correlation coefficient (r) and the p value of each correlation are indicated (n=7; *p<0.05).

FIG. 11A-FIG. 11D show that LysoSM accumulates to similar extent in brain, liver, and plasma of ASMko mice and does not induce toxicity in cultured ASMko neurons. FIG. 11A depicts mean±SEM LysoSM levels in brain, liver, and plasma from wt and ASMko mice. Data are expressed as percentage of wt samples (n=6; ***p<0.001; ****p<0.0001). FIG. 11B shows mean±SEM of total SM levels in cultured wt neurons incubated with LysoSM expressed as percentage of vehicle treated cultures (n=3 independent cultures). FIG. 11C shows mean±SEM cellular viability measured by MTT in cultured wt neurons treated with vehicle or LysoSM and expressed as percentage of living cells with respect to vehicle treated cultures (n=3 independent cultures; p>0.05). FIG. 11D shows the linear regression of LysoSM levels in plasma (x-axis) and brain or liver (y-axis) of wt and ASMko mice at 4.5 months of age. The correlation coefficient (r) and the p value of each correlation are indicated (n=7; *p<0.05).

FIG. 12A-FIG. 12D show that ASM has a preference to degrade SM16:0, which accumulates in lysosomes when ASM is pharmacologically inhibited. FIG. 12A depicts mean±SEM levels of fluorescence in arbitrary units with respect to nmol of SMb or SM16:0 metabolized by Smase C (NSmase analogue) activity (n=3 independent experiments). FIG. 12B shows mean±SEM levels of fluorescence in arbitrary units with respect nmol of SMb or SM16:0 metabolized by ASM activity (n=3 independent experiments; slope analysis *p<0.05). FIG. 12C shows pSM16:0 colocalization with the lysosomal marker Lamp1 in wt neurons treated or not with the ASM inhibitor desipramine (n=3 independent cultures). FIG. 12D shows mean±SEM Mander's coefficient of colocalization between pSM16:0 and Lamp1 in wt neurons treated or not with the ASM inhibitor desipramine (n=3 independent cultures).

FIG. 13A shows total SM levels in brain, liver, and plasma of ASMko mice injected with vehicle or with rhASM. FIG. 13B shows SM species in brain, liver, and plasma of ASMko mice injected with vehicle or with rhASM. FIG. 13C shows LSM levels in brain, liver, and plasma of ASMko mice injected with vehicle or with rhASM. FIG. 13D shows SM OH levels in brain, liver, and plasma of ASMko mice injected with vehicle or with rhASM. FIG. 13E shows levels of m/z805 in brain, liver, and plasma of ASMko mice injected with vehicle or with rhASM. FIG. 13F shows peak area ratio of m/z805 levels in brain, liver, and plasma of WT vs ASMko mice. FIG. 13G shows percent of m/z805 levels in brain, liver, and plasma of WT vs ASMko mice. FIG. 13H shows correlation of m/z805 levels in the brain/liver vs plasma of WT vs ASMko mice. FIG. 13I shows peak area ratio of m/z805 levels in WT vs ASMko mice at ages 2 months, 4 months, and 6 months. FIG. 13J shows m/z805 levels in brain/liver vs plasma of WT vs ASMko mice at ages 2 months, 4 months, and 6 months.

### DETAILED DESCRIPTION

Provided herein are compositions and methods of determining an increased risk of a lysosomal storage disease, diagnosing the same, methods of treating the same, and compositions for use in any of the aforementioned methods. Also provided are compositions comprising shRNA targeting a ceramide synthase sequence and compositions and methods of delivering the same. In aspects, also provided are compositions and methods of newborn screening of lysosomal storage diseases.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the” include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “effective amount” or “therapeutically effective amount” refers to the amount of an agent that is sufficient to effectuate beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “subject” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile; fish, or the like.

As used herein, “treating” or “treat” describes the management and care of a subject for the purpose of combating a disease, condition, or disorder and includes the administration of the Tn3 scaffold used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., RA). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### Lysosomal Storage Disease

In aspects, Lysosomal storage diseases (LSDs) are inborn errors of metabolism characterized by the accumulation of substrates in excess in various organs' cells due to the defective functioning of lysosomes. In aspects, LSDs are caused by mutations in the genes encoding a lysosomal enzyme.

Various LSDs are contemplated herein. In aspects, LSDs are classified according to the accumulated substrate. Representative examples of LSDs are provided in Table 1. In aspects, a lysosomal storage disease of the disclosure is selected from one or more of those listed in Table 1.

TABLE 1
Exemplary LSDs

| Accumulated Substrate | Disease |
|---|---|
| Sphingolipidosis | Tay Sachs disease |
| | Sandhoff disease |
| | GM2 activator deficiency |
| | Niemann-Pick diseases A and B (acid |
| | sphingomyelinase deficiency (ASMD)), and |
| | C (transmembrane protein defect C1, |
| | soluble non-enzymatic protein defect C2) |
| | Gaucher disease types 1, 2, and 3 |
| | Fabry disease |
| | Metachromatic leukodystrophy |
| | Globoid leukodystrophy |
| | GM1 gangliosidosis types 1, 2, and 3 |
| | Multiple sulfatase deficiency |
| Oligosaccharidosis | Alfa mannosidosis |
| | Schindler disease |
| | Aspartylglucosaminuria |
| | Fucosidosis |
| Mucopolysaccharidosis | Hurler syndrome |
| | Scheie syndrome |
| | Hurler-Scheie syndrome |
| | Hunter syndrome |
| | SanFilippo syndrome A, B, C, and D |
| | Morquio syndrome A and B |
| | Maroteaux-Lamy syndrome |
| | Sly syndrome |
| Neuronal ceroid lipofuscinosis | CLN 1 through CLN 14 |
| Sialic acid disorders | Galactosialidosis |
| | Infantile sialic acid storage disease |
| | Salla disease |
| | Sialuria |
| Mucolipidosis | Sialidosis I and II |
| | I-cell disease |
| | Pseudo-Hurler-Polydystrophy |
| | Mucolipidosis IV |
| Other | Lysosomal Acid lipase deficiency infantile |
| | and childhood/adult types |
| | Pompe disease |
| | Danon disease |
| | Cystinosis |

In aspects, a LSD comprises Sphingolipidosis, Oligosaccharidosis, Mucopolysaccharidosis, Neuronal ceroid lipofuscinosis, Sialic acid disorder, Mucolipidosis, and combinations thereof. In aspects, a LSD comprises Sphingolipidosis and is selected from the group consisting of: Tay Sachs disease, Sandhoff disease, GM2 activator deficiency, Niemann-Pick disease, Gaucher disease, Fabry disease, Metachromatic leukodystrophy, Globoid leukodystrophy, GM1 gangliosidosis, Multiple sulfatase deficiency, and any combination thereof. In aspects, a LSD comprises Niemann-Pick disease. In Niemann-Pick disease SM accumulation impairs neuronal processes selected from the group consisting of: autophagy, calcium hemostasis, and synaptic activity. In aspects, a Niemann-Pick disease is of a type selected from the group consisting of: A, B (acid sphingomyelinase deficiency (ASMD)), C, and combinations thereof. In aspects, a Niemann-Pick disease is type A (acid sphingomyelinase deficiency (ASMD)). In aspects, a Niemann-Pick disease is type B (acid sphingomyelinase deficiency (ASMD)). In aspects, a Niemann-Pick disease is type C.

In aspects, a Niemann-Pick disease is type A (acid sphingomyelinase deficiency (ASMD))). Type A Niemann-Pick comprises ASM activity of less than about 2%. In aspects, type A comprises onset in infancy. In aspects, type A comprises neurological involvement. In aspects, type A comprises infantile neurovisceral ASMD. In aspects, a LSD is type A/B Niemann-Pick Disease. Type A/B comprises ASM activity of less than about 10%. In aspects, type A/B comprises childhood onset. In aspects type A/B comprises neurologic involvement. In aspects, type A/B comprises chronic neurovisceral ASMD. In aspects, a Niemann-Pick disease is type B. Type B comprises ASM activity of less than about 10%. In aspects, type B comprises onset from childhood to adulthood. In aspects, type B comprises reduced or no neurological involvement. In aspects, type B comprises chronic visceral ASMD.

In aspects, a LSD comprises Sphingolipidosis. Sphingomyelin (SM) is one of the most abundant sphingolipids in cellular membranes. It comprises a polar headgroup, phosphocholine, a hydrophobic backbone, and ceramide. Its chemical properties, together with its significant interactions with cholesterol, can contribute to formation of membrane microdomains that serve as signaling platforms. Moreover, SM metabolism produces important second messengers (e.g., ceramides) for signal transduction. Synthesis and degradation of SM can be controlled by a complex array of enzymes, which in mammalian cells comprise different isoforms of ceramide synthases (CerS), sphingomyelin synthases (SMS) and sphingomyelinases (SMases). These enzymes can show differences in their levels of expression depending on the tissue, cell type and subcellular compartment. In aspects, numerous SM species exist that differ in the length and degree of saturation of their fatty acids, and these different species can have distinct subcellular localization, metabolism and properties. SM can be enriched at the plasma and synaptic membranes of neurons. Besides its involvement in synapses by modulating neurotransmitter receptor physiology and dendritic spine dynamics, SM contributes to other important physiological processes in neurons such as the establishment of axonal polarity, autophagy, and calcium homeostasis. In aspects, alterations in neuronal SM levels lead to severe neurological diseases. Among them is the infantile neurovisceral form of the acid sphingomyelinase deficiency (ASMD), also called Niemann Pick type A. Loss of function mutations in the sphingomyelin phosphodiesterase 1 (SMPD1) gene encoding acid sphingomyelinase (ASM) cause severe neurological disease, neurodegeneration and death in the first years of life. Intermediate and nonneurologic forms of the disease also have been described (type A/B and type B, respectively).

In aspects, an LSD comprises Sphingolipidosis and comprises acid sphingomyelinase deficiency (ASMD). ASMD is a lysosomal storage disorder caused by mutations in the SMPD1 gene encoding for the acid sphingomyelinase (ASM). Aberrantly high SM levels are a hallmark of all ASMD cells. In aspects, increased SM levels are directly responsible for many pathological phenotypes in neurons (Toledano-Zaragoza and Ledesma, 2019), Intravenous infusion of recombinant ASM has proven successful to treat peripheral ASMD disease in mouse models (Miranda et al., 2000) and patients (Wasserstein et al., 2018; Wasserstein et al., 2015). However, the recombinant ASM does not cross the blood brain barrier (BBB) and therefore leaves the neurological phenotype unaddressed. Most, if not all, studies in ASMD have referred to the deleterious effects of the increase in the levels of total SM in the brain. However, analysis of potential changes in the different SM species and metabolic enzymes, and their contribution to toxicity in ASMD is still lacking. This information may reveal new strategies aimed at reducing specifically certain species, and provide new insights regarding the underlying CNS pathology in this disease. In addition, altered levels of these species in CSF and/or plasma could become indicators for brain disease and therapy response in ASMD, which are currently lacking

### Determination of Risk of Lysosomal Storage Disease (LSD)

Provided herein are compositions and methods of determining risk of developing, having, and/or dying from a lysosomal storage disease. In aspects, the risk is of developing a LSD. In aspects, the risk is of having or being afflicted by a LSD. In aspects, the risk is of dying of a LSD or disease secondary to the LSD.

Methods for determining risk of a LSD can comprise determining an increased level of a substrate (e.g., a substrate of Table 1). In aspects, a substrate comprises sphingomyelin. In aspects, the determining of risk comprises determining an increased level of sphingomyelin in a subject, or sample from the subject, as compared to a baseline level of the subject, or an otherwise comparable healthy subject.

In aspects, the risk determination comprises an in vitro test, an in vivo test, or both. In aspects, an in vitro test comprises a laboratory test. In aspects, an in vivo test comprises a physical examination. Exemplary laboratory tests comprise chemistry, molecular genetics, human leukocyte antigen, and cytogenetic testing.

In aspects, a test comprises a chemistry test evaluating a level of sphingomyelin in circulating blood. In aspects, a method comprises determining a level of sphingomyelin. In aspects, a method comprises determining an increased level of sphingomyelin as compared to a baseline level or as compared to a level in a healthy comparable subject. In aspects, a method comprises obtaining a sample from a subject and determining a level of sphingomyelin in the sample.

A test can evaluate sphingolipids including but not limited to: sphingoid bases, sphingosine and dihydrosphingosine, their 1-phosphates (S1P and dhS1P), molecular species (Cn-) of ceramide (Cer), sphingomyelin (SM), hexosylceramide (HexCer), lactosylceramide (LacCer), and Cer 1-phosphate (Cer1P). SM, LacCer, HexCer, Cer, and Cer1P. In aspects, the sphingolipid is SM, and the SM is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof. In aspects, the SM is sphingomyelin 16:0.

In aspects, a SM is sphingomyelin 16:0. In aspects, sphingomyelin 16:0 is the SM species showing the highest relative increase in a brain of a subject as compared to other species. sphingomyelin 16:0 can target lysosomes and/or show neuronal toxicity.

In aspects, the level of the substrate (e.g., sphingomyelin) is increased by at least about 5 fold, 10 fold, 25 fold, 50 fold, 75 fold, 100 fold, 125 fold, 150 fold, 175 fold, 200 fold, 225 fold, 250 fold, 275 fold, 300 fold, 325 fold, 350 fold, 375 fold, 400 fold, 425 fold, 450 fold, 475 fold, 500 fold, 525 fold, 550 fold, 575 fold, 600 fold, 625 fold, 650 fold, 675 fold, 700 fold, 725 fold, 750 fold, 775 fold, 800 fold, 825 fold, 850 fold, 875 fold, 900 fold, 925 fold, 950 fold, 975 fold, or up to about 1000 fold as compared to a baseline level or as compared to a level in a healthy comparable subject. In aspects, the level is increased by at least about 0%, 25%, 50%, 75%, 100%, 125%, 150%, 175%, 200%, 225%, 250%, 275%, 300%, 325%, 350%, 375%, 400%, 425%, 450%, 475%, or up to about 500% as compared to a baseline level or as compared to a level in a healthy comparable subject. In aspects, the level is increased by at least about 5-10 fold, 5-20 fold, 10-30 fold, 15-30 fold, 20-50 fold, 30-80 fold, 50-75 fold, 50-100 fold, 45-120 fold, 100-200 fold, 150-250 fold, 200-275 fold, 250-350 fold, 300-400 fold, or 350-500 fold as compared to a baseline level or as compared to a level in a healthy comparable subject. In aspects, the level is increased by at least about 5-10%, 5-20%, 10-30%, 15-30%, 20-50%, 30-80%, 50-75%, 50-100%, 45-120%, 100-200%, 150-250%, 200-275%, 250-350%, 300-400%, or 350-500% as compared to a baseline level or as compared to a level in a healthy comparable subject.

In aspects, a method of determining risk of a LSD comprises an in vitro test determining the presence or level of a biomarker. Exemplary biomarkers comprise oxysterols, Lysosphingomyelin-509 and other lysosphingolipids, phosphocholines, bile acids, and combinations thereof. In aspects, a biomarker comprises a phosphocholine, and the phosphocholine comprises m/z805. In aspects, a biomarker comprises SM16:0.

In aspects, a method of determining risk of a LSD comprises determining the presence of a phosphocholine is a subject thought to be at risk of or thought of having an LSD. In aspects, the phosphocholine is m/z805.

In aspects, a method of determining risk of a LSD comprises an in vitro test determining ASM enzyme activity in peripheral blood lymphocytes or cultured skin fibroblasts. In aspects, subjects with Niemann-Pick disease type A or B have less than about 10% of normal ASM activity compared to healthy subjects.

In aspects, a method of determining risk of a LSD comprises an in vivo test comprising a physical exam. A physical exam can elucidate whether a subject has enlargement of an organ such as a liver and/or spleen enlargement. A physical exam can also elucidate eye movement difficulties. In aspects, a physical exam can be used to identify one or more of ataxia, intellectual disability, developmental delay, cognitive impairment or decline, dystonia, dementia, schizophrenia, interstitial lung disease, classic cherry-red spot of the retina, and combinations thereof.

In aspects, a method of determining risk of a LSD comprises a molecular genetic test. A molecular genetic test may determine the presence of a genetic mutation associated with a LSD. In aspects, a molecular genetic test associated with Niemann-Pick type A or B evaluates SMPD1 for a mutation. A SMPD1 gene mutation can be selected from the group consisting of: R496L, L302P, fsP330, deltaR608, and combinations thereof. Niemann-Pick disease type C (NP-C) is associated with autosomal recessive mutations in NPC1 or NPC2. Molecular genetic tests can be conducted by way of traditional sequencing methods (e.g., Sanger sequencing of genomic DNA (gDNA) or complementary DNA (cDNA)), next generation sequencing (NGS), complementary genetic testing (e.g., Array comparative genomic hybridization (CGH)), full-gene sequencing, and the like. In aspects, a molecular genetic test comprises SMPD1 sequencing analyzing the entire coding region of the SMPD1 gene. In aspects, molecular genetic testing comprises SMPD1 Deletion/Duplication Analysis and/or SMPD1 known familial mutation testing. The diagnosis of ASM deficiency and/or risk of LSD can be established by detection of either biallelic pathogenic variants in SMPD1 on molecular genetic testing and/or residual ASM enzyme activity that is less than 10% of controls (in peripheral blood lymphocytes or cultured skin fibroblasts as previously described).

Methods of determining risk can be performed on a subject or a sample thereof. Subjects of the disclosure can be mammalian subjects. Mammals may be dogs, cats, cows, horses, sheep, pigs, hamsters, mice, squirrels, and primates such as monkeys, gorillas, chimpanzees, bonobos, and humans. In aspects, a mammalian subject is a human. In aspects, a human is a pediatric subject. In aspects, a subject is an infant. An infant can be from 0-12 months of age. In aspects, a subject is a newborn infant. A newborn infant can be from 0 up to about 1 month of age. In aspects, a newborn is from about 0-3 days, 0-7 days, 0-14 days, 7-14 days, or 7-30 days old. In aspects, a newborn is from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or up to about 30 days old. In aspects, a subject is an adult.

In aspects, a method comprises determining risk of a LSD in a newborn. In aspects, the determining is completed after birth. In aspects, the determining is completed within about 24, 48, or 72 hours of birth. In aspects, a method comprises determining risk within about 0 hrs., 2 hrs., 4 hrs., 6 hrs., 8 hrs., 10 hrs., 12 hrs., 14 hrs., 16 hrs., 18 hrs., 20 hrs., 22 hrs., 24 hrs., 26 hrs., 28 hrs., 30 hrs., 32 hrs., 34 hrs., 36 hrs., 38 hrs., 40 hrs., 42 hrs., 44 hrs., 46 hrs., 48 hrs., 50 hrs., 52 hrs., 54 hrs., 56 hrs., 58 hrs., 60 hrs., 62 hrs., 64 hrs., 66 hrs., 68 hrs., 70 hrs., 72 hrs., 74 hrs., 76 hrs., 78 hrs., 80 hrs., 82 hrs., 84 hrs., 86 hrs., 88 hrs., 90 hrs., 92 hrs., 94 hrs., 96 hrs., 98 hrs., 100 hrs., 102 hrs., 104 hrs., 106 hrs., 108 hrs., 110 hrs., 112 hrs., 114 hrs., 116 hrs., 118 hrs., 120 hrs., 122 hrs., 124 hrs., 126 hrs., 128 hrs., 130 hrs., 132 hrs., 134 hrs., 136 hrs., 138 hrs., 140 hrs., 142 hrs., 144 hrs., 146 hrs., 148 hrs., 150 hrs., 152 hrs., 154 hrs., 156 hrs., 158 hrs., 160 hrs., 162 hrs., 164 hrs., 166 hrs., 168 hrs., 170 hrs., 172 hrs., 174 hrs., 176 hrs., 178 hrs., 180 hrs., 182 hrs., 184 hrs., 186 hrs., 188 hrs., 190 hrs., 192 hrs., 194 hrs., 196 hrs., 198 hrs., or 200 hrs. of birth.

A sample of the disclosure can be obtained from any part of the body. In aspects, a sample is obtained from the central nervous system of a subject. In aspects, a sample is from an organ. In aspects, a sample is from the circulatory system. In aspects, a sample comprises a bodily fluid. A bodily fluid sample means a bodily fluid obtained from a subject or a sample derived from the bodily fluid. In aspects, the bodily fluid sample may be blood, serum, plasma, lymph fluid, tissue fluids such as interstitial fluid, intercellular fluid, interstitial fluid, amniocentesis, chorionic villus sampling, and the like, and may be body cavity fluid, serosal fluid, pleural fluid, ascites fluid, capsular fluid, cerebrospinal fluid (CSF), joint fluid (synovial fluid), and aqueous humor of the eye (aqueous humor). In aspects, the bodily fluid may be digestive fluid such as saliva, gastric juice, bile, pancreatic juice, intestinal fluid, etc., and may be sweat, tears, runny nose, urine, semen, vaginal fluid, amniotic fluid, milk, and the like.

In aspects, a sample is a blood sample. A blood sample can be peripheral blood. In aspects, a sample is obtained from blood and is processed to obtain peripheral blood mononuclear cells (PBMCs). In aspects, a blood sample can be obtained via blood draw, finger prick, heel prick, and combinations thereof.

In aspects, a sample comprises a nucleic acid. A nucleic acid can comprise DNA or RNA.

Any of the aforementioned samples may be from a healthy subject, a subject with a particular disease (e.g., LSD), or from a subject suspected of suffering from a LSD disease.

In aspects, a sample may be used as the stock solution, or it may be a liquid diluted or concentrated from the stock solution. In aspects, a sample may include an additive. In aspects, a sample can be frozen.

### Agents

Provided herein are agents and compositions comprising the same. In aspects, an agent is effective in reducing a level of a substrate associated with a LSD. Exemplary substrates are described herein. In aspects, a substrate comprises sphingomyelin. Agents described herein can be used to reduce levels of sphingomyelin by way of targeting a metabolic enzyme that comprises a ceramide synthase. In aspects, an agent targets a ceramide synthase. In aspects, an agent targets SM 16:0. Provided are also neuromodulatory agents and methods of determining whether an agent is neuromodulatory.

In aspects, the metabolic enzyme comprises acid sphingomyelinase. Acid sphingomyelinase can generate a ceramide synthase via degradation of SM. Ceramide is a metabolite of the sphingolipid family, structurally comprised of a sphingoid base-generally 18 carbon dihydrosphingosine or sphingosine—with a variable length fatty acyl side-chain. Ceramides form the lipid backbone to which a diverse array of headgroup structures are conjugated, forming sphingomyelin (SM), glucosyl- and galactosylceramide (HexCer), gangliosides, and globosides. In aspects, an agent of the disclosure targets a ceramide synthase selected from the group consisting of: CerS4, CerS5, and CerS6. In aspects, the ceramide synthase is CerS4. In aspects, the ceramide synthase is CerS5. In aspects, the ceramide synthase is CerS6. In aspects, a subject with a LSD comprises high levels of a ceremide, such as CerS5 as compared to an otherwise comparable subject without the LSD.

Agents of the disclosure can function by inhibiting a provided metabolic enzyme, silencing an RNA encoding a metabolic enzyme, or genomically disrupting a nucleic acid encoding the metabolic enzyme. In aspects, an agent functions by silencing the RNA encoding the metabolic enzyme (e.g., acid sphingomyelinase). In aspects, genetic silencing of a ceramide synthase ameliorates brain pathology of a subject having a LSD.

Exemplary agents comprise shRNA, guide RNA (gRNA) (e.g., CRISPR systems), protein inhibitors, and the like. In aspects, an agent is a shRNA. In aspects, an agent is a CRISPR system comprising a gRNA. In aspects, an agent is a protein inhibitor.

In aspects, an agent comprises a shRNA. In aspects, an shRNA comprises a stem-loop and microRNA-adapted shRNA. A simple stem-loop shRNA can be transcribed under the control of an RNA Polymerase III (Pol III) promoter. The 50-70 nucleotide transcript forms a stem-loop structure consisting of about 19 to 29 bp region of double-strand RNA (the stem) bridged by a region of predominantly single-strand RNA (the loop) and a dinucleotide 3′ overhang. The simple stem-loop shRNA is transcribed in the nucleus and enters the RNAi pathway similar to a pre-microRNA. In aspects, an shRNA can be a longer (>250 nucleotide) microRNA-adapted shRNA comprising a design that more closely resembles native pri-microRNA molecules, and consists of a shRNA stem structure which may include microRNA-like mismatches, bridged by a loop and flanked by 5′ and 3′ endogenous microRNA sequences. The microRNA-adapted shRNA, like the simple stem-loop hairpin, is also transcribed in the nucleus but can enter the RNAi pathway earlier similar to an endogenous pri-microRNA.

In aspects, provided is an shRNA that comprises a stem-loop. In aspects, an shRNA comprises a sense sequence, a loop sequence, and an antisense sequence. In aspects, an shRNA comprises a sense sequence, loop sequence, and antisense sequence comprising at least about 80%, 85%, 90%, 95%, 96%, 97%, 98%, 99%, or 100% identity to SEQ ID NO: 23, TCAAGAG, and SEQ ID NO: 24, respectively. In aspects, an shRNA binds to a target that comprises a sequence that binds or is complementary to one of SEQ ID NO: 23 and/or SEQ ID NO: 24.

shRNA of the disclosure can be delivered to a cell by way of DNA-based delivery. In aspects, DNA-based delivery employs plasmids, lentiviral vectors, adenoviral vectors, and the like. In aspects, an adenoviral vector is utilized.

A viral vector can comprise an adenoviral vector, an adeno-associated viral vector (AAV), a lentiviral vector, a retroviral vector, a portion of any of these, or any combination thereof. In aspects, a vector can comprise an AAV vector. An AAV vector can be modified to include a modified VP protein (such as an AAV vector modified to include a VP1 protein, VP2 protein, or VP3 protein). In an aspect, an AAV vector is a recombinant AAV (rAAV) vector. Suitable AAV vectors can be selected from any AAV serotype or combination of serotypes. For example, an AAV vector can be any one of: AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rhlO, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68, or any combination thereof. In aspects, an AAV is of a serotype selected from the group consisting of: AAV2, AAV5, AAV6, AAV8, AAV9, a portion thereof, a fusion product thereof, and any combination thereof. In aspects, an AAV is of serotype AAV9.

In aspects, a vector is selected based on its natural tropism. In aspects, a vector serotype is selected based on its ability to cross the blood brain barrier. AAV9 and AAV10 have been shown to cross the blood brain barrier to transduce neurons and glia. In aspects, an AAV vector is AAV2, AAV5, AAV6, AAV8, or AAV9. In aspects, an AAV vector is a chimera of at least two serotypes. In aspects, an AAV vector can be self-complementary. In aspects, an AAV vector can comprise an inverted terminal repeat. In aspects, an AAV vector can comprise an inverted terminal repeat (scITR) sequence with a mutated terminal resolution site. In aspects, rep, cap, and ITR sequences can be mixed and matched from all the of the different AAV serotypes provided herein.

In aspects, a suitable AAV vector can be further modified to encompass modifications such as in a capsid or rep protein. Modifications can also include deletions, insertions, mutations, and combinations thereof. In aspects, a modification to a vector is made to reduce immunogenicity to allow for repeated dosing. In aspects, a serotype of a vector that is utilized is changed when repeated dosing is performed to reduce and/or eliminate immunogenicity.

In aspects, an AAV vector can comprise from 2 to 6 copies of an agent per viral genome. In aspects, an AAV vector can comprise from 1 to 2, from 1 to 3, from 1 to 4, from 1 to 5, from 1 to 6, from 1 to 7, from 1 to 8, from 1 to 9, from 1 to 10, from 2 to 3, from 2 to 4, from 2 to 5, from 2 to 6, from 2 to 7, from 2 to 8, from 2 to 9, or from 2 to 10 copies of an agent per viral genome. In aspects, an AAV vector can comprise 1, 2, 3, 4, 5, 6, 7, 8, 9, or 10 copies of an agent per viral genome. In aspects, an AAV vector can comprise from 1 to 5, from 1 to 10, from 1 to 15, from 1 to 20, from 1 to 25, from 1 to 30, from 1 to 35, from 1 to 40, from 1 to 45, or from 1 to 50 copies of an agent per viral genome.

Vectors provided herein can be utilized to transfect a target cell. Target cells can be found in any tissues and organs of the body. In aspects, a target cell is found in a tissue or organ implicated in a LSD.

Provided are also neuromodulatory agents and methods of identifying and making the same. In aspects, provided is a method of determining if an agent is neuromodulatory, the method comprising determining a blood plasma concentration of sphingomyelin in a subject treated with an agent, wherein a decrease in the blood plasma concentration, as compared to a baseline level, is indicative of the efficacy of the compound as a neuromodulatory agent. In aspects, an agent is neuromodulatory when the blood plasma concentration of sphingomyelin is decreased by at least about 5 fold, 10 fold, 25 fold, 50 fold, 75 fold, 100 fold, 125 fold, 150 fold, 175 fold, 200 fold, 225 fold, 250 fold, 275 fold, 300 fold, 325 fold, 350 fold, 375 fold, 400 fold, 425 fold, 450 fold, 475 fold, or up to about 500 fold as compared to a baseline level of the subject or an otherwise comparable subject lacking the treatment. In aspects, the method is performed on a non-human mammal. In aspects, the sphingomyelin is sphingomyelin 16:0.

In aspects, the method comprises assaying a plurality of different agents. In aspects, a method comprises selecting an agent from a plurality of agents thought to be neuromodulatory. In aspects, an agent that is identified using a method disclosed herein is formulated into a pharmaceutical composition. In aspects, the agent identified in a method described herein is used in a method of treatment of a LSD. In aspects, the LSD comprises acid sphingomyelinase deficiency.

### Methods of Prevention or Treatment of LSDs

Provided herein are also methods of preventing or treating a LSD. In aspects, a method comprises administering an agent in an amount effective to reduce a level of a substrate. Exemplary substrates are provided herein. In aspects, a substrate comprises sphingomyelin.

Prevention and/or treatment can comprise obtaining a desired pharmacologic effect, physiologic effect, or any combination thereof. In aspects, a treatment can reverse an adverse effect attributable to a LSD. In aspects, a treatment can stabilize a LSD. In aspects, a treatment can delay progression of a LSD. In aspects, a treatment can cause regression of a LSD. In aspects, a treatment can at least partially prevent the occurrence of a LSD. In aspects, a treatment's effect can be measured. In aspects, measurements can be compared before and after administration of an agent of the disclosure. For example, a subject can have testing results obtained prior to treatment compared to results after treatment to show regression of disease and/or improvement. In aspects, a subject can have an improved blood test result (e.g., reduced sphingomyelin) after treatment compared to a blood test before treatment. In aspects, measurements can be compared to a standard or control sample from a comparable healthy subject.

In aspects, a method comprises an administration of a composition described herein. In aspects, after the administration, a subject comprises: (a) at least a 1-fold reduced formation of sphingomyelin as compared to an otherwise comparable subject lacking the administering, as measured by: a brain scan, a blood test, or both. In aspects, the reduction is measured by brain scan. In aspects, a method comprises an administration of a composition described herein. In aspects, after the administration, a subject comprises an at least a 1-fold increase in acid sphingomyelinase, as compared to an otherwise comparable subject lacking the administering, as determined a brain scan, a blood test, or both. In aspects, the reduction or the increase is measured by a blood test. In aspects, the reduction is measured by a brain scan and a blood test.

In aspects, after administration of a composition described herein, a subject experiences at least a 5 fold, 10 fold, 25 fold, 50 fold, 75 fold, 100 fold, 125 fold, 150 fold, 175 fold, 200 fold, 225 fold, 250 fold, 275 fold, 300 fold, 325 fold, 350 fold, 375 fold, 400 fold, 425 fold, 450 fold, 475 fold, 500 fold, 525 fold, 550 fold, 575 fold, 600 fold, 625 fold, 650 fold, 675 fold, 700 fold, 725 fold, 750 fold, 775 fold, 800 fold, 825 fold, 850 fold, 875 fold, 900 fold, 925 fold, 950 fold, 975 fold, or up to about 1000 fold reduced formation of sphingomyelin as compared to an otherwise comparable subject lacking the administration. In aspects, the sphingomyelin is sphingomyelin 16:0.

In aspects, after administration of a composition described herein, a subject experiences at least a 5 fold, 10 fold, 25 fold, 50 fold, 75 fold, 100 fold, 125 fold, 150 fold, 175 fold, 200 fold, 225 fold, 250 fold, 275 fold, 300 fold, 325 fold, 350 fold, 375 fold, 400 fold, 425 fold, 450 fold, 475 fold, 500 fold, 525 fold, 550 fold, 575 fold, 600 fold, 625 fold, 650 fold, 675 fold, 700 fold, 725 fold, 750 fold, 775 fold, 800 fold, 825 fold, 850 fold, 875 fold, 900 fold, 925 fold, 950 fold, 975 fold, or up to about 1000 fold increase in acid sphingomyelinase as compared to an otherwise comparable subject lacking the administration.

In aspects, administration of an agent of the disclosure is effective in reducing or eliminating a LSD in a subject in need thereof. In aspects, a symptom of the LSD is reduced or eliminated after administration of an agent of the disclosure. In aspects, a symptom of a LSD is reduced at least about or at most about 1, 2, 3, 4, 5, 6, 7, 12, 14, 16, 18, 20, 25, 30, 35, 40, 50, 60, or 70 days post agent administration. In aspects, an agent of the disclosure is effective in reducing or eliminating a LSD for at least about or at most about 1, 2, 3, 4, 5, 6, 7, 12, 14, 16, 18, 20, 25, 30, 35, 40, 50, 60, or 70 days post agent administration. In aspects, the LSD comprises acid sphingomyelinase deficiency.

Provided are methods of preventing or treating acid sphingomyelinase deficiency comprising administering an AAV particle of the disclosure to a subject in need thereof, thereby preventing or treating the acid sphingomyelinase deficiency. In aspects, the administering is effective in reducing an amount of sphingomyelin 16:0 in the subject in need thereof as compared to an otherwise comparable subject lacking the administering.

### Pharmaceutical Composition

Compositions and methods provided herein can utilize pharmaceutical compositions. The compositions described throughout can be formulated into a pharmaceutical and be used to treat a human or mammal, in need thereof, diagnosed with a LSD, or at risk of developing or dying of LSD. In aspects, pharmaceutical compositions can be used prophylactically.

In aspects, a pharmaceutical composition comprises an inhibitor of a ceramide synthase. In aspects, a pharmaceutical composition comprises a short hairpin RNA (shRNA) targeting a ceramide synthase. In aspects, a pharmaceutical composition comprises a guide RNA that targets a nucleic acid sequence encoding a ceramide synthase. In aspects, a pharmaceutical composition comprises a guide RNA that targets a ribonucleic acid sequence encoding acid sphingomyelinase. In aspects, a pharmaceutical composition comprises an AAV particle comprising a nucleic acid sequence encoding any of the aforementioned compositions.

In aspects, a subject of the disclosure receives standard-of-care for a LSD. Exemplary therapeutics that can be administered comprise: Zavesca and/or rhASM. In aspects, a subject receives recombinant human ASM (rhASM).

In aspects, a composition of the disclosure is administered as part of a therapeutic regimen that comprises at least one standard-of-care therapy. In aspects, a subject is administered a recombinant acid sphingomyelinase. A therapeutic regimen of the disclosure can comprise one or more administrations of a composition of the disclosure. In aspects, a therapeutic regiment comprises administration of a composition of the disclosure and recombinant acid sphingomyelinase. In aspects, a therapeutic regiment comprises administration of a composition of the disclosure comprising a shRNA (e.g., an shRNA targeting a ceramide synthase) and recombinant acid sphingomyelinase. In aspects, a subject was pre-treated with recombinant acid sphingomyelinase prior to administration of a composition of the disclosure (e.g., a shRNA).

Any of the disclosed pharmaceutical compositions can comprise a pharmaceutically acceptable excipient, carrier, or diluent. Exemplary carriers and excipients can include dextrose, sodium chloride, sucrose, lactose, cellulose, xylitol, sorbitol, malitol, gelatin, PEG, PVP, and any combination thereof.

A pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The pharmaceutical compositions of the disclosure may be administered to a subject via a variety of routes known in the art. Exemplary routes of administering of such pharmaceutical compositions include intrathecal, oral, mucosal, topical, transdermal, inhalation, parenteral, sublingual, buccal, rectal, vaginal, and intranasal. In aspects, a pharmaceutical composition of the disclosure is formulated to be administered by routes selected from the group consisting of oral, topical, transdermal, inhalation, parenteral, sublingual, buccal, rectal, vaginal, and intranasal routes. The term parenteral, as used herein, includes subcutaneous injections, intravenous, intramuscular, intrasternal injection or infusion techniques. In aspects, an administration is performed intrathecally, intraocularly, intravitreally, retinally, intravenously, intramuscularly, intraventricularly, intracerebrally, intracerebrally, intracerebroventricularly, intraperenchymally, subcutaneously, or any combination thereof. In aspects, an administration comprises an intrathecal administration.

In aspects, compositions disclosed herein can be in unit dose forms or multiple-dose forms. For example, a pharmaceutical composition described herein can be in unit dose form. Unit dose forms, as used herein, refer to physically discrete units suitable for administration to human or non-human subjects (e.g., pets, livestock, non-human primates, and the like) and packaged individually. Each unit dose can contain a predetermined quantity of an active ingredient(s) that can be sufficient to produce the desired therapeutic effect in association with pharmaceutical carriers, diluents, excipients, or any combination thereof. Examples of unit dose forms can include ampules, syringes, and individually packaged tablets and capsules.

In certain cases, the pharmaceutical compositions of the present disclosure can be prepared by techniques known to those skilled in the art. General considerations in the formulation and/or manufacture of pharmaceutical compositions may be found, for example, in Remington: The Science and Practice of Pharmacy 21st ed., Lippincott Williams & Wilkins, 2005 (incorporated herein by reference in its entirety).

### Kits

Any of the compositions described herein may be comprised in a kit. In a non-limiting example, an agent and reagents to generate the agent can be provided in a kit. In aspects, kit components are provided in suitable container means.

The current disclosure is described with reference to the following examples. The examples are illustrative only, and the disclosure should in no way be construed as being limited to these examples but rather should be construed to encompass any and all variations which become evident as a result of the teachings provided herein.

### NUMBERED EMBODIMENTS

Notwithstanding the appended claims, the following numbered embodiments also form part of the instant disclosure.
1. A method of determining an increased risk of a lysosomal storage disease in a subject, the method comprising determining an increased level of sphingomyelin in the subject as compared to a baseline level of the subject or an otherwise comparable healthy subject.
2. The method of embodiment 1, wherein the increased level of sphingomyelin is detected in a blood sample of the subject.
3. The method of embodiment 1, wherein the increased level of sphingomyelin is detected in a sample from the central nervous system of the subject.
4. The method of embodiment 3, wherein the sample from the central nervous system comprises cerebrospinal fluid.
5. The method of any one of embodiments 1-4, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.
6. The method of embodiment 5, wherein the sphingomyelin is sphingomyelin 16:0.
7. The method of any one of embodiments 1-6, wherein the lysosomal storage disease comprises acid sphingomyelinase deficiency.
8. The method of any one of embodiments 1-7, wherein the subject is pediatric.
9. The method of embodiment 8, wherein the subject is an infant.
10. The method of embodiment 9, wherein the infant is a newborn infant.
11. The method of embodiment 10, wherein the determining is completed within about 24 hours, 48 hours, or 72 hours of birth.
12. A method of preventing or treating a lysosomal storage disease, the method comprising administering an agent in an amount effective to reduce a level of sphingomyelin in the brain of a subject in need thereof, wherein the agent targets a metabolic enzyme that comprises a ceramide synthase.
13. The method of embodiment 12, wherein the ceramide synthase is selected from the group consisting of CerS4, CerS5, and CerS6.
14. The method of embodiment 13, wherein the ceramide synthase is CerS5.
15. The method of any one of embodiments 12-14, wherein the metabolic enzyme consists of the ceramide synthase.
16. The method of any one of embodiments 12-15, wherein the agent inhibits the metabolic enzyme, silences an RNA encoding the metabolic enzyme, or genomically disrupts a nucleic acid encoding the metabolic enzyme.
17. The method of embodiment 16, wherein the agent silences the RNA encoding the metabolic enzyme.
18. The method of embodiment 17, wherein the agent comprises an adeno-associated virus (AAV) expressing a shRNA that silences the RNA encoding the metabolic enzyme.
19. The method of embodiment 18, wherein the shRNA comprises a sense sequence, loop sequence, and antisense sequence comprising at least about 80% identity to SEQ ID NO: 23, TCAAGAG, and SEQ ID NO: 24, respectively.
20. The method of embodiment 19, wherein the shRNA comprises the sense sequence, loop sequence, and antisense sequence of SEQ ID NO: 23, TCAAGAG, and SEQ ID NO: 24, respectively.
21. The method of any one of embodiments 18-20, wherein the AAV is of a serotype selected from the group consisting of: AAV2, AAV5, AAV6, AAV8, AAV9, a portion thereof, a fusion product thereof, and any combination thereof.
22. The method of embodiment 21, wherein the AVV is of serotype AAV9.
23. The method of any one of embodiments 12-22, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.
24. The method of embodiment 23, wherein the sphingomyelin is sphingomyelin 16:0.
25. The method of any one of embodiments 12-24, wherein the lysosomal storage disease comprises acid sphingomyelinase deficiency.
26. The method of any one of embodiments 12-25, wherein the level is reduced by at least about 1-fold, 5-fold, 20-fold, or 50-fold as compared to an otherwise comparable method lacking the administering.
27. The method of any one of embodiments 12-26, wherein the subject is a pediatric subject.
28. The method of any one of embodiments 12-26, wherein the subject is an adult.
29. The method of any one of embodiments 12-28, wherein the subject is concurrently administered recombinant acid sphingomyelinase.
30. The method of any one of embodiments 12-29, wherein the subject was previously administered recombinant acid sphingomyelinase.
31. A composition comprising: a) an inhibitor of a ceramide synthase; b) a short hairpin RNA (shRNA) targeting a ceramide synthase; c) a guide RNA that targets a nucleic acid sequence encoding a ceramide synthase; or d) a guide RNA that targets a ribonucleic acid sequence encoding acid sphingomyelinase.
32. The composition of embodiment 31, wherein the composition comprises the shRNA.
33. The composition of embodiment 32, wherein the shRNA comprises a sense sequence, loop sequence, and antisense sequence comprising at least about 80% identity to SEQ ID NO: 23, TCAAGAG, and SEQ ID NO: 24, respectively.
34. The composition of embodiment 33, wherein the shRNA comprises the sense sequence, loop sequence, and antisense sequence of SEQ ID NO: 23, TCAAGAG, and SEQ ID NO: 24, respectively.
35. An AAV particle comprising a sequence encoding a short hairpin RNA (shRNA) targeting a ceramide synthase.
36. The AAV particle of embodiment 35, wherein the particle is of a serotype selected from the group comprising: AAV2, AAV5, AAV6, AAV8, AAV9, a portion thereof, a fusion product thereof, and any combination thereof.
37. The AAV particle of embodiment 36, wherein the particle comprises serotype AAV9.
38. The AAV particle of any one of embodiments 35-37, wherein the shRNA comprises a sense sequence, loop sequence, and antisense sequence comprising at least about 80% identity to SEQ ID NO: 23, TCAAGAG, and SEQ ID NO: 24, respectively.
39. The AAV particle of embodiment 38, wherein the shRNA comprises the sense sequence, loop sequence, and antisense sequence of SEQ ID NO: 23, TCAAGAG, and SEQ ID NO: 24, respectively.
40. The composition or the AAV particle of any one of embodiments 31-39, wherein the ceramide synthase is selected from the group consisting of: CerS4, CerS5, and CerS6.
41. The composition or the AAV particle of embodiment 40, wherein the ceramide synthase is CerS5.
42. A plurality of AAV particles comprising the AAV particle of any one of embodiments 35-39.
43. The plurality of embodiment 42, wherein the plurality is in unit dose form. 44. A pharmaceutical composition comprising:

a) the composition of any one of embodiments 31-34, the AAV particle of any one of embodiments 35-39, or the plurality of any one of embodiments 42-43; and b) a pharmaceutically acceptable excipient, carrier, or diluent.
45. A method of treating or preventing a disease or a condition in a subject in need thereof, the method comprising: administering to the subject the pharmaceutical composition of embodiment 44 thereby treating or preventing the disease or the condition.
46. The method of embodiment 45, wherein the administering is intrathecally, intraocularly, intravitreally, retinally, intravenously, intramuscularly, intraventricularly, intracerebrally, intracerebellarly, intracerebroventricularly, intraperenchymally, subcutaneously, or a combination thereof.
47. The method of embodiment 46, wherein the administering is intrathecal.
48. The method of any one of embodiments 45-47, wherein the disease or condition is a lysosomal storage disease.
49. The method of embodiment 48, wherein the lysosomal storage disease comprises acid sphingomyelinase deficiency.
50. The method of any one of embodiments 45-49, wherein after the administering, the subject comprises: a) at least a 1-fold reduced formation of sphingomyelin as compared to an otherwise comparable subject lacking the administering, as measured by: a brain scan, a blood test, or both; or b) at least a 1-fold increase in acid sphingomyelinase, as compared to an otherwise comparable subject lacking the administering, as determined a brain scan, a blood test, or both.
51. An AAV particle comprising a sequence encoding a short hairpin RNA (shRNA) targeting a ceramide synthase, wherein the shRNA targets a sequence that is bound by or complementary to at least one of SEQ ID NO: 23 or SEQ ID NO: 25.
52. The AAV particle of embodiment 51, wherein the AAV particle is of serotype AAV9.
53. The AAV particle of any one of embodiments 51-52, wherein the ceramide synthase is CerS5.
54. A method of preventing or treating acid sphingomyelinase deficiency comprising administering the AAV particle of any one of embodiments 51-53 to a subject in need thereof, thereby preventing or treating the acid sphingomyelinase deficiency.
55. The method of embodiment 54, wherein the administering is effective in reducing an amount of sphingomyelin 16:0 in the subject in need thereof as compared to an otherwise comparable subject lacking the administering.
56. A method of determining whether an agent is neuromodulatory, the method comprising determining a blood plasma concentration of sphingomyelin in a subject treated with a neuromodulatory agent, wherein a decrease in the blood plasma concentration as compared to a baseline level is indicative of the efficacy of the compound as a neuromodulatory agent.
57. The method of embodiment 56, wherein when the decrease is detected, the subject continues treatment with the neuromodulatory agent.
58. The method of any one of embodiments 56-57, wherein the subject has a lysosomal storage disease.
59. The method of embodiment 58, wherein the lysosomal storage disease comprises acid sphingomyelinase deficiency.
60. The method of any one of embodiments 56-59, wherein the subject is pediatric.
61. The method of any one of embodiments 56-60, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.
62. The method of embodiment 61, wherein the sphingomyelin is sphingomyelin 16:0.
63. A method of selecting a biomarker, the method comprising: administering a composition comprising ASM to a subject; and detecting a level of a lipid in the subject after the administering, wherein when the level of the lipid decreases in a liver of the subject but not a brain or plasma, the lipid is selected.
64. The method of embodiment 63, wherein the lipid comprises a phosphocholine.
65. The method of embodiment 64, wherein the phosphocholine comprises m/z805.
66. A composition comprising m/z805.

### EXAMPLES

### Example 1—Sphingomyelin (SM) 16:0 is the SM Species with the Highest Relative Increase in ASMko Compared to WT Mouse Brains and Neurons

### Method of Quantification of SM Species in the Brain and Liver of ASMko Mice

Mouse brain and liver samples were homogenized in water (1 g wet tissue/4 mL). SM was extracted from 50 μL of homogenate or 50 μL of plasma in the presence of SM (17:0) as internal standard using Bligh-Dyer lipid extraction (Bligh and Dyer, 1959). Sample analysis was performed in a Shimadzu 20AD HPLC system coupled to a 6500QTRAP+ mass spectrometer (AB Sciex, Framingham, MA) operated in positive multiple reaction monitoring mode. Data processing was conducted with Analyst 1.6.3 (AB Sciex). Data were reported as the peak area ratios of the analytes in sample to the internal standard.

### Method of Quantification of SM Species in Cultured Neurons

Sample analysis was performed in an Acquity ultraperformance liquid chromatography (UPLC) system (Waters, USA) connected to a time-of-flight (TOF; LCT Premier XE) detector controlled with Waters/Micromass MassLynx software.

### Method of Quantification of Total SM by Enzymatic Assay

SM levels were measured according to protocols modified from (Hojati and Jiang, 2006). Lipid extracts were dried in the presence of Thesit, and SM was converted into peroxide by incubation with sphingomyelinase (Smase C from Merck; #S9396 or ASM from Sino Biological; #50749-M08B), alkaline phosphatase, and choline oxidase. Peroxide was measured fluorimetrically in the presence of peroxidase and homovanillic acid (Van Veldhoven et al., 1997).

### Results

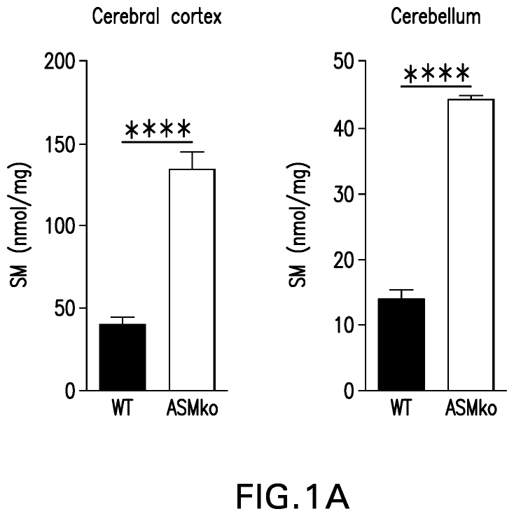

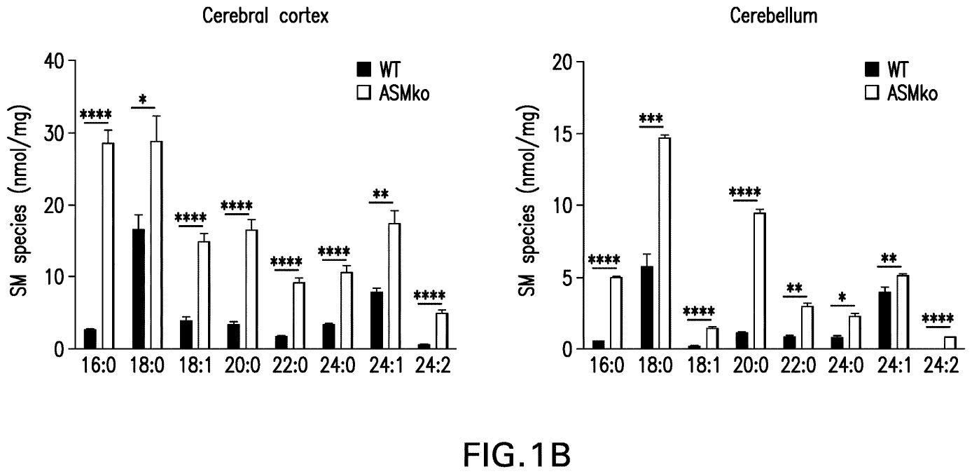

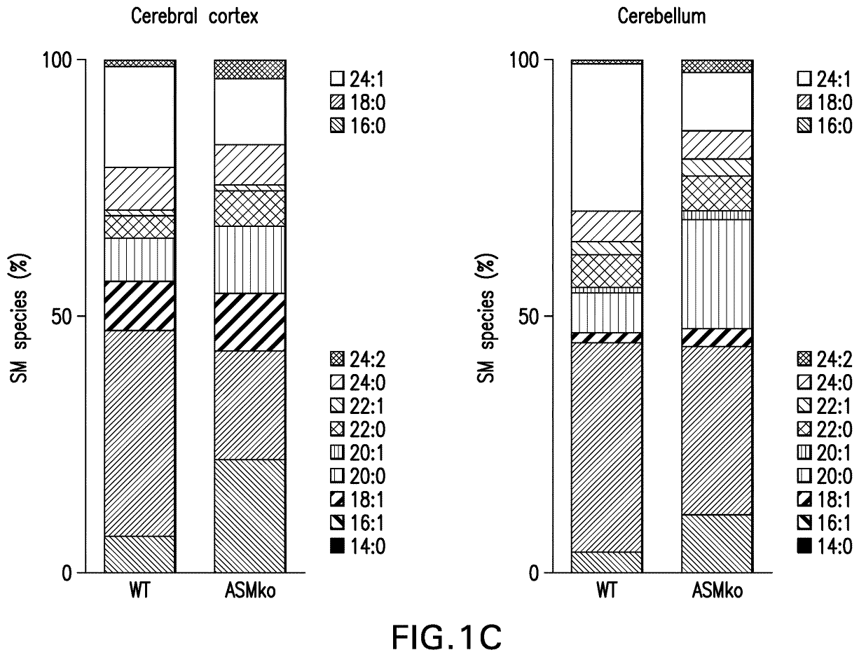

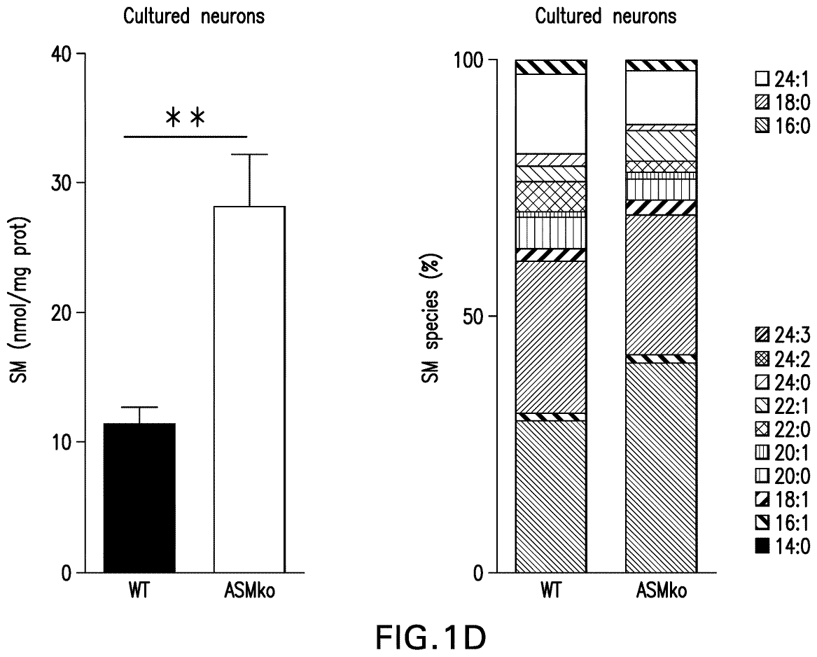

Levels of total SM and of different SM species were analyzed by Liquid Chromatography/Mass Spectrometry (LC/MS) in the cerebellum and cortex of wt and ASMko mice at 4.5 months of age. At this age, neurological symptoms of the disease are evident including motor and cognitive impairment (Arroyo et al., 2014; Horinouchi et al., 1995). The results confirmed a similar increment of total SM levels in the cortex and cerebellum of the ASMko compared to wt mice (3.3-fold and 3.2-fold, respectively) (FIG. 1A). However, while the levels of all SM species were increased, the extent of this increase was variable (FIG. 1B and FIG. 1C). Intriguingly, the SM species that increased less were the most abundant in wt conditions: SM18:0 (1.7-fold increase in the ASMko cortex and 2.6-fold in cerebellum) and SM24:1 (2.2-fold cortex; 1.3-fold cerebellum). In contrast, the amount of SM16:0, which exists in minor amounts in the wt situation, showed a dramatic increase (10.8-fold cortex; 8.6-fold cerebellum) (FIG. 1B). Analyses of the data in terms of relative amounts of these SM species indicated that SM18:0 and SM24:1 accounted for 60% and 70% of total SM in the wt mouse cortex and cerebellum, respectively. These percentages decreased to 33% and 44% of total SM in the cortex and cerebellum of the ASMko mouse brain (FIG. 1C). In contrast, the relative amount of SM16:0 with respect to total SM increased from less than 6% and 4% in wt cortex and cerebellum to 20% and 11%, respectively, in the ASMko mice (FIG. 1C). To confirm whether the changes observed in brain extracts also occurs in neurons, LC/MS analysis was performed in primary cultures of cortical neurons derived from wt and ASMko mice (FIG. 1D). Total SM levels showed a 2.5-fold increase in the ASMko neurons compared to wt. Analysis of the relative amounts of SM species showed similar trends than in the brain. Thus, while SM18:0 and SM24:1 reduced their prevalence (by 3% and 5% respectively) the relative abundance of SM16:0 increased (by 12%) in the ASMko neurons compared to wt (FIG. 1D).

### Example 2—SM16:0 is the SM Species that Accumulates the Most and Shows Highest Toxicity in Cultured Neurons

### Method of Lysotracker Staining

Lysotracker Red DND99 (Thermo Fisher Scientific) was added to wt primary cultured neurons at 1 μM and incubated for 20 min at 37° C. Cells were then fixed in 4% PFA and mounted using Prolong Gold Antifade (Invitrogen). Images were taken on a confocal LSM710 microscope (Zeiss).

### Method of LAMP 1 Surface Staining in Cultured Neurons

ASMko and wt cultured neurons at 14DIV were washed with PBS and fixed with 2% PFA in PBS and 0.12 M sucrose for 8 min. Immunofluorescence was performed using a conventional protocol in the absence of detergent to avoid cell permeabilization.

### Method of Reactive Oxygen Species Determination

To assess reactive oxygen species (ROS), cultured neurons were incubated for 20 min with dihydrorhodamine 123 (10 μM, DHR: Molecular Probes, Carlsbad, CA, USA). The fluorescence product of DHR, rhodamine-123, was detected at excitation and emission wavelengths of 500 and 536 nm, respectively. Images of cultured neurons were captured on a confocal microscope (LSM710; Zeiss).

### Method of Cell Viability Assay

Cell viability was evaluated using the MTT (methylthiazolyldiphenyltetrazoliumbromide, Sigma) assay. Briefly, 3×10⁴ neurons were plated in 96-well plates coated with poly-L-lysine (0.1 mg/ml). At DIV14 1 mM MTT was added to the wells and incubated for 4 h. Then, the supernatant was discarded, and 500 μl DMSO was added to the wells. The absorbance was measured at 570 nm using a microplate reader (FLUOstar Optima, BMG Labtech). Cells treated with DMSO were used as a control.

### Results

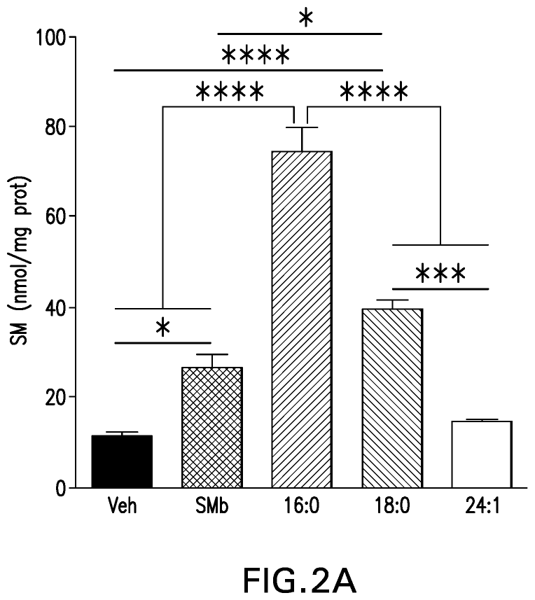

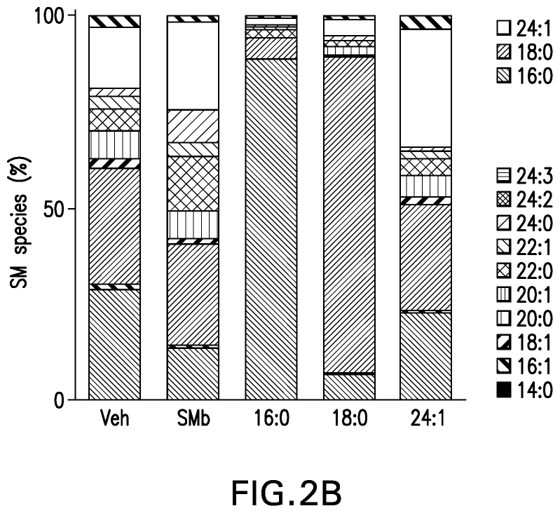

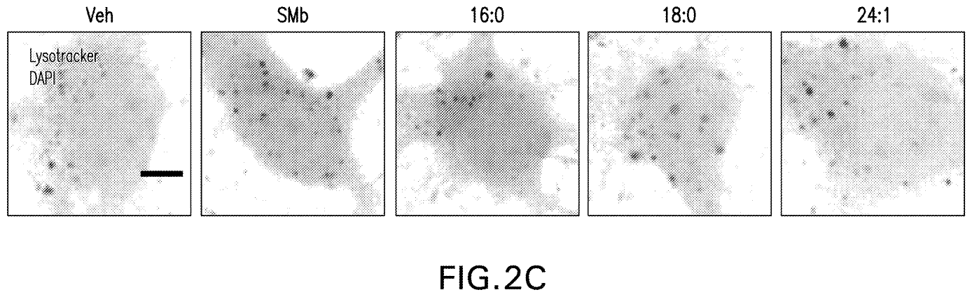

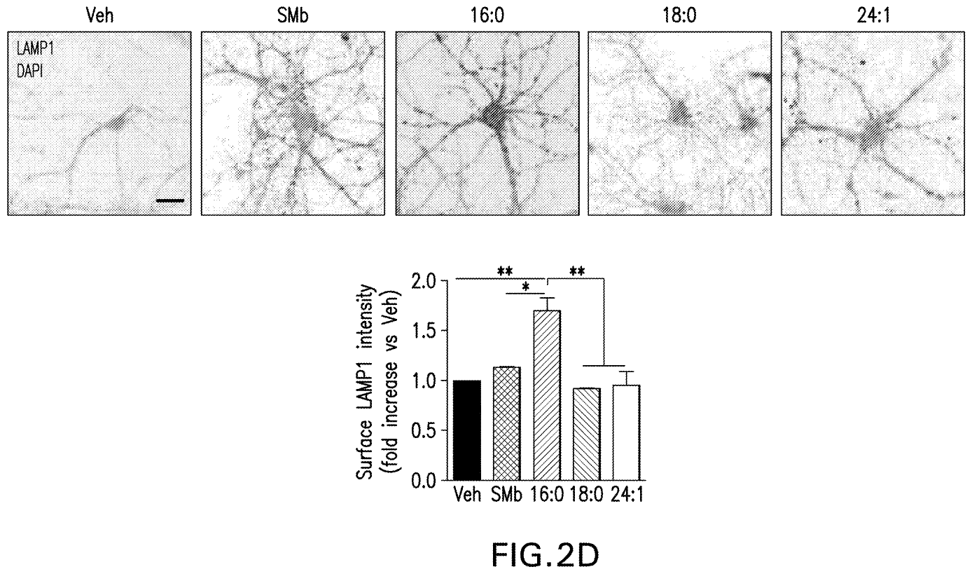

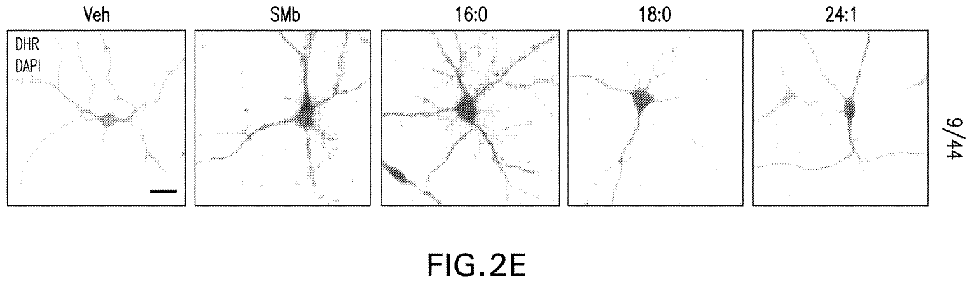

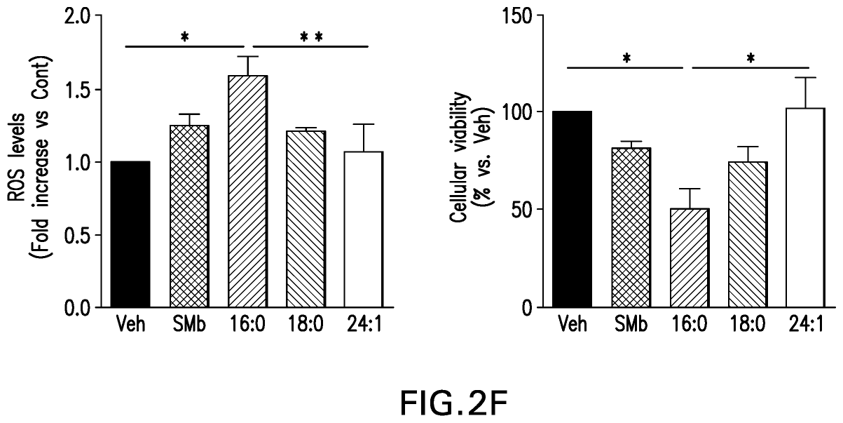

A study was done to determine the impact of the different increments of individual SM species in ASMko mice and cells on their specific accumulation in neurons. Cortical neurons from wt mice were cultured for 48 hr with 40 μM of either total SM from bovine brain (SMb, containing similar SM species proportions than total SM from mouse brain); or individual SM16:0; SM18:0 or SM24:1. LC/MS analyses revealed that while addition of SMb, SM16:0 and SM18:0 resulted in a significant increase of total SM levels (2.3-fold; 6.6-fold and 3.5-fold, respectively), in the wt cells, the increment contribution of SM24:1 to the total SM content was not significant (FIG. 2A). LC/MS quantification assessed the effect of each treatment on the relative abundance of the added SM species. Incubation with SM16:0 and SM18:0 drastically raised the relative abundance of each of these species to 88% and 82% of the total (FIG. 2B). Incubation with SM24:1 increased the prevalence of this species to 30%, while addition of SMb reduced the relative abundance of SM18:0 and SM16:0 by 4% and 15%, respectively, while increased that of SM24:1 by 8% (FIG. 2B).

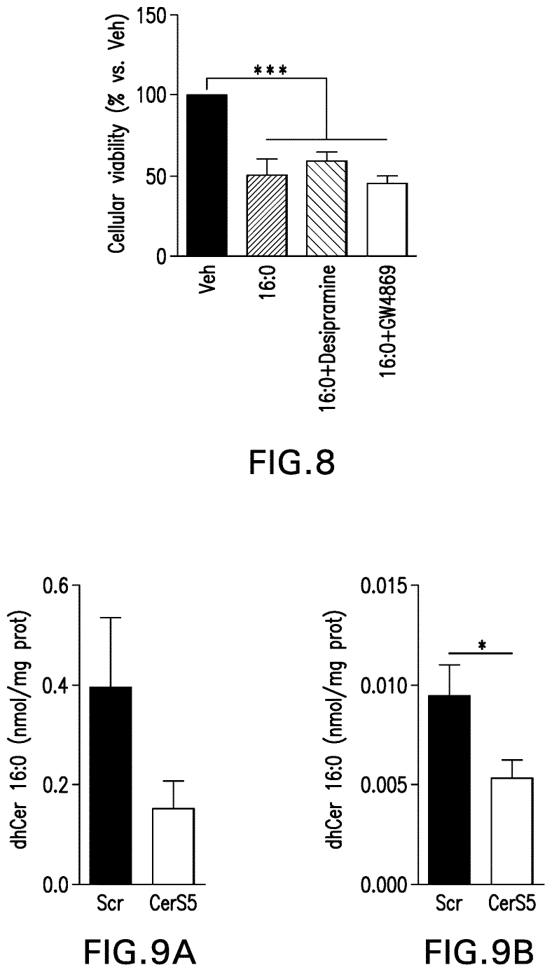

Accumulation of lipids in lysosomes is a pathological hallmark in ASMD cells that classifies this disease as a lysosomal storage disorder. To investigate the effect of the different SM species in these organelles, several lysosomal-related parameters were measured. To analyze lysosomal permeabilization, the lysotracker red dye, which fluoresces in acidic environments was utilized. SM16:0 was the SM species that significantly changed the staining of the lysotracker probe from the dotty pattern observed in the vehicle treated cells to a more diffuse cytosolic pattern, which is indicative of lysosomal permeabilization (FIG. 2C). This effect was also observed after addition of SMb but not of SM18:0 or SM24:1. SM16:0 was also the only SM species that induced lysosomal exocytosis as indicated by the increased surface staining of the lysosomal associated membrane protein LAMP1 in non-permeabilized neurons (FIG. 2D). Since high levels of SM also can induce oxidative stress, reactive oxygen species (ROS) was evaluated by dihydrorhodamine 123 (DHR) staining in wt neurons incubated with the different SM species. SM16:0 induced the highest elevation in ROS levels (1.6-fold) compared to the other SM species (FIG. 2E). To determine whether different SM species could have distinct toxicity effects on neurons, cell viability was analyzed by the MTT technique. While addition of SMb and SM18:0 reduced neuronal survival by 19% and 25%, respectively, SM16:0 had a more severe impact causing the death of 50% of the seeded cells. In contrast, SM24:1 had no deleterious effects on neuronal survival (FIG. 2F). To confirm that the cell death observed upon SM16:0 addition is due to this SM species, and not to its conversion into ceramide, we added SM16:0 in the presence of inhibitors for the acid and neutral sphingomyelinases (desipramine and GW4869, respectively). In these conditions neuronal death was not prevented as determined by the MTT assay (FIG. 8).

### Example 3—SM16:0 Accumulates in the Endolysosomal Compartment of Cultured Neurons

### Method of Immunofluorescence Assay

Cultured neurons at 14 DIV were fixed in 4% PFA 0.12M sucrose and incubated overnight with primary antibodies and subsequently for 1 h with Alexa 488- or Alexa 555-conjugated secondary antibodies. Images were taken using a confocal microscope LSM710 (Zeiss). The Mander's coefficient for pSM 16:0 co-localization with Thy1; PSD95; TOM20; EEA1; LAMP1; GM130 was determined by using the JACOP plugin. Mouse brains were dissected, fixed in 4% PFA 0.12M sucrose, and cryoprotected for 24 h in 30% sucrose phosphate buffer saline. The tissue was then frozen in Tissue-Tek optimal cutting temperature compound (Sakura Finetek, Torrance, CA, USA), and 40-μm sagittal sections were obtained with a cryostat (CM 1950 Ag Protect freezing: Leica, Solms, Germany). The sections were incubated overnight at 4° C. with the primary antibodies and then with the corresponding Alexa-conjugated secondary antibodies. Finally, the sections were incubated for 10 min with DAPI (Merck), washed, and mounted with ProLong Gold Antifade (Thermo Fisher). Images were obtained on a confocal LSM710 microscope (Zeiss) and quantified using the Fiji software.

### Propargyl SM

Cultured neurons were treated with 40 μM propargyl SM (pSM 16:0) (Merck; #860711P) for 48 hours at DIV12. After immunofluorescence protocol, explained above, click reaction was performed for the attachment of fluorescent reporter (Sulfo-Cy3-Azide; Jena Biosciences; #CLK-AZ119-1) to pSM 16:0.

### Results

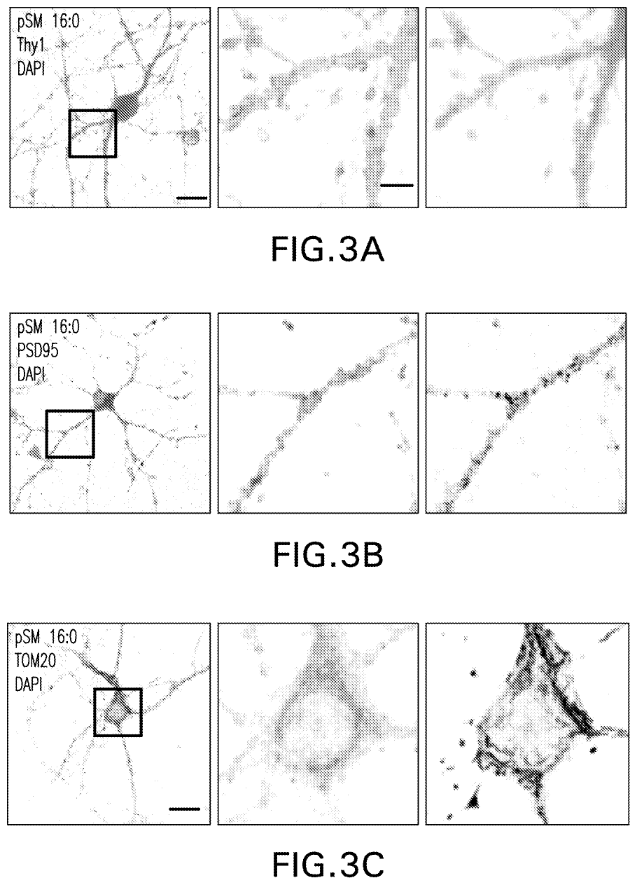

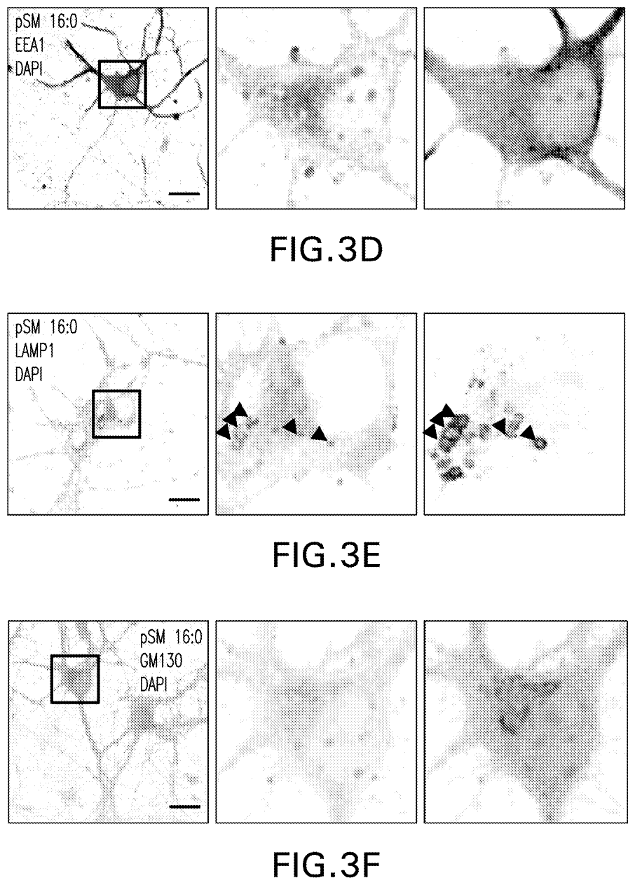

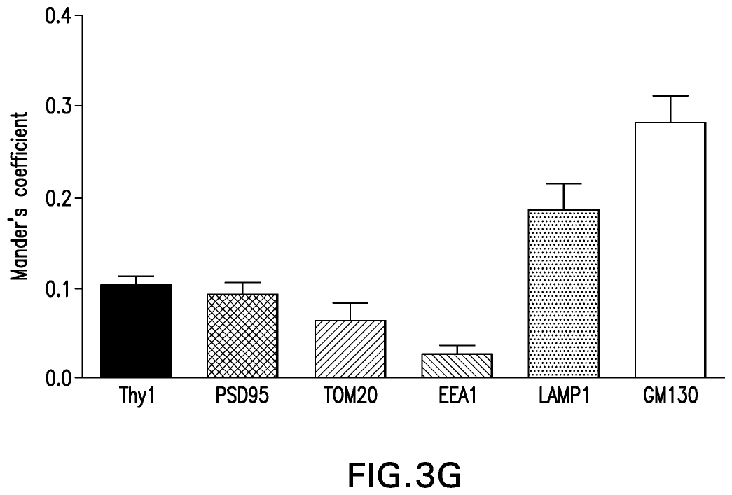

The surprising effects of SM16:0 on lysosomes compared to other SM species supported a hypothesis that this lipid targets these organelles. To explore this possibility, we took advantage of the existence of the analogue propargyl SM16:0 (pSM16:0), which is linked to an alkyne group and can be traced by click chemistry and fluorescence microscopy. We added pSM16:0 for 48 hours to cultured wt neurons and performed double immunofluorescence with markers of different cellular compartments. Analysis of the Mander's coefficient indicated a low degree of co-localization of pSM16:0 with the plasma membrane marker Thy-1 (FIG. 3A), the synaptic marker PSD95 (FIG. 3B), the mitochondrial marker TOM20 (FIG. 3C) or the early endosome marker EEA1 (FIG. 3D). In contrast, pSM16:0 showed a high colocalization with the endolysosomal marker LAMP1 (FIG. 3E) and the Golgi apparatus marker GM130 (FIG. 3F). These results supported that late endosome and lysosomes are a target organelle for SM16:0 in neurons.

### Example 4—Altered Gene Expression of SM Metabolic Enzymes in the Brain of ASMko Mice

### Method of Quantitative RT-PCR

Total RNA from wt and ASMko cerebellum and cortex was extracted with TRIzol Reagent (Ambion/RNA Life Technologies Co.) following the manufacturer instructions. RNA was quantified by absorbance at 260 nm using a NanoDrop ND-100 (Themo Fisher Scientific Inc.). Retrotranscription to first-strand cDNA was performed using RevertAid H Minus First-Strand cDNA Synthesis Kit (Thermo Fisher Scientific Inc.). Briefly, 10 ng of synthesised cDNA were used to perform fast qPCR using GoTaq qPCR Master Mix (Promega Co., Madison, WI, USA) in ABI PRISM 7900HT SDS (Applied Biosystems; Life Technologies Co.) following manufacturer instructions. Primer sequences, shown in Table 2, were used at 0.5 μM final concentration. Three housekeeping genes (Gapdh, GusB and Pgk1) were used as endogenous controls.

TABLE 2
Primer Sequences

| SEQ | | |
|---|---|---|
| ID | | |
| NO | ID | Sequence 5′-3′ |
| 1 | mouse CerS1: forward | GCATCAGTGCCCTGTACTGT |
| 2 | mouse CerS1: reverse | GACCAACTTCTACCCTGGGC |
| 3 | mouse CerS2: forward | AATAACAATCATCCTAAGAAT |
| 4 | mouse CerS2: reverse | TAGACTCCTCATATCCTT |
| 5 | mouse CerS3: forward | CAACTTTCGAGGGTGGGTCT |
| 6 | mouse CerS3: reverse | TTCTTCTTCCCAAAGCGCCA |
| 7 | mouse CerS4: forward | TTGCCTTAGTCTTCTTCT |
| 8 | mouse CerS4: reverse | GATGGAGTCATACACAGA |
| 9 | mouse CerS5: forward | TCTACACTCCTGTGACTA |
| 10 | mouse CerS5: reverse | AGCCAATAACCTTACTGAA |
| 11 | mouse CerS6: forward | TACTGCTCTGACGACTTG |
| 12 | mouse CerS6: reverse | GCTGTTGCTGCTATTCTC |
| 13 | mouse Sgms1: forward | CGTTGGAATCTTCTGTAT |
| 14 | mouse Sgms1: reverse | CTTGTGGTGATGTAGTAG |
| 15 | mouse Sgms2: forward | AAAGGCCACCTGTCTGTCC |
| 16 | mouse Sgms2: reverse | GGCCTGACCAATGCTCTCTT |
| 17 | mouse Smpd2: forward | TGGGACATCCCCTACCTGAG |
| 18 | mouse Smpd2: reverse | TAGGTGAGCGATAGCCTTTGC |
| 19 | mouse Smpd3: forward | ACACGACCCCCTTTCCTAATA |
| 20 | mouse Smpd3: reverse | GGCGCTTCTCATAGGTGGTG |
| 21 | mouse Smpd4: forward | CACACTAGCCTCTTGAAGCGA |
| 22 | mouse Smpd4: reverse | TGTAGAACCTCCAACTTGGCAT |

### Results

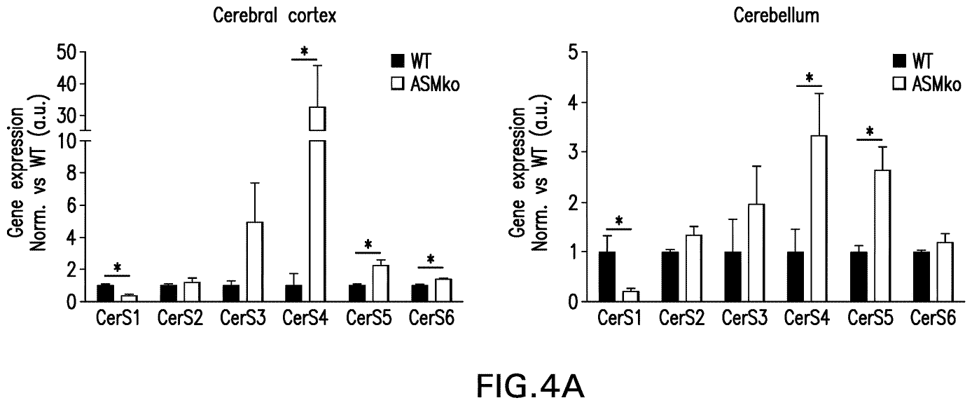

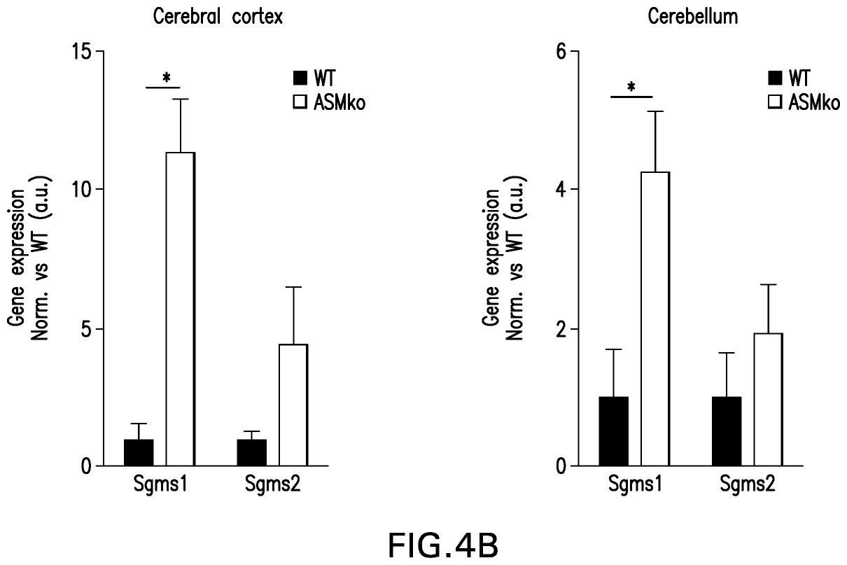

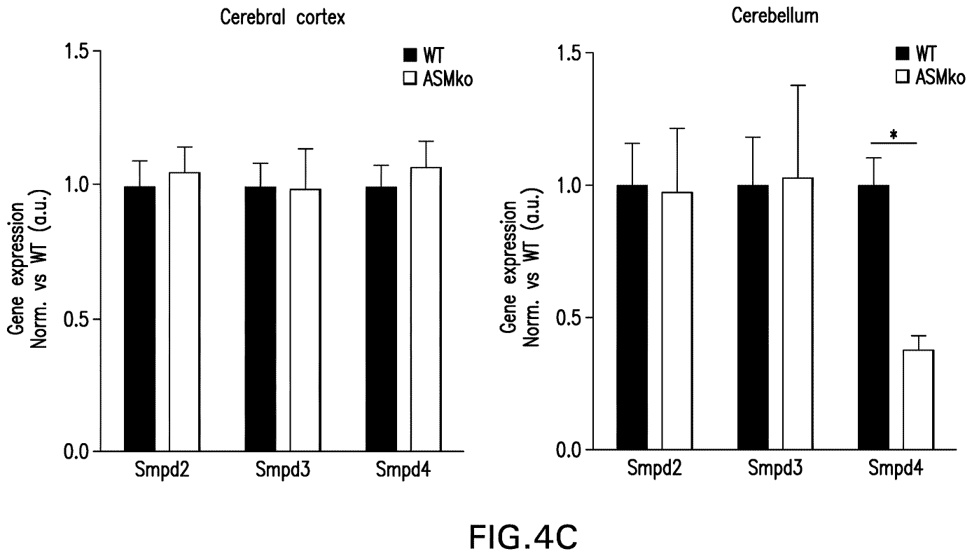

The gene expression of SM anabolic and catabolic enzymes was analyzed. Quantitative PCR (qPCR) of the genes encoding for CerS1-6, SMS1-2 and Smases2-4 was performed in cortical and cerebellar extracts of wt and ASMko mice at 4.5 months of age (FIG. 4A-FIG. 4C). In the ASMko cortex, CerS1 gene expression was 2.8-fold reduced while CerS4, 5 and 6 increased by 32.6, 2.2 and 1.3-fold, respectively; CerS2 and CerS3 did not change significantly compared to wt (FIG. 4A). In the ASMko cerebellum we observed similar reduction for CerS1 (4.5-fold) and increase for CerS4 (3.3-fold) and CerS5 (2.6-fold), while the levels of CerS 2, 3 and 6 did not change (FIG. 4A).

Surprisingly, the expression of SMS genes was not decreased despite the accumulation of SMs. In contrast, an increase of SMS1 was observed in both cortical (11.3-fold) and cerebellar (4.2-fold) extracts of ASMko mice compared to wt (FIG. 4B). We did not find significant changes in the expression of the genes encoding for Smases, except for the 2.7-fold reduction of SMPD4 in the cerebellum (FIG. 4C).

### Example 5—Genetic Silencing of CerS5 Prevents Lysosomal Damage and Oxidative Stress in Cultured ASMko Neurons

### Method of In Vitro Adenoviral Infection

wt cultured neurons were treated with adenovirus containing shRNA-scramble or shRNA-CerS5 at a multiplicity of infection (MOI)=20. Viral infection was done at 6DIV and for 24 hours.

Viral particles containing the following shRNA sequences were used for the genetic silencing of CerS5: shRNAsense sequence “GCATGTGGAGATTCACTTATT” (SEQ ID NO: 23); loop sequence and “TCAAGAG”; shRNA antisense sequence “AATAAGTGAATCTCCACATGC” (SEQ ID NO: 24). These sequences were under the expression of the HI promoter sequence and included SalI, NheI and AgeI restriction enzymes sites.

### Results

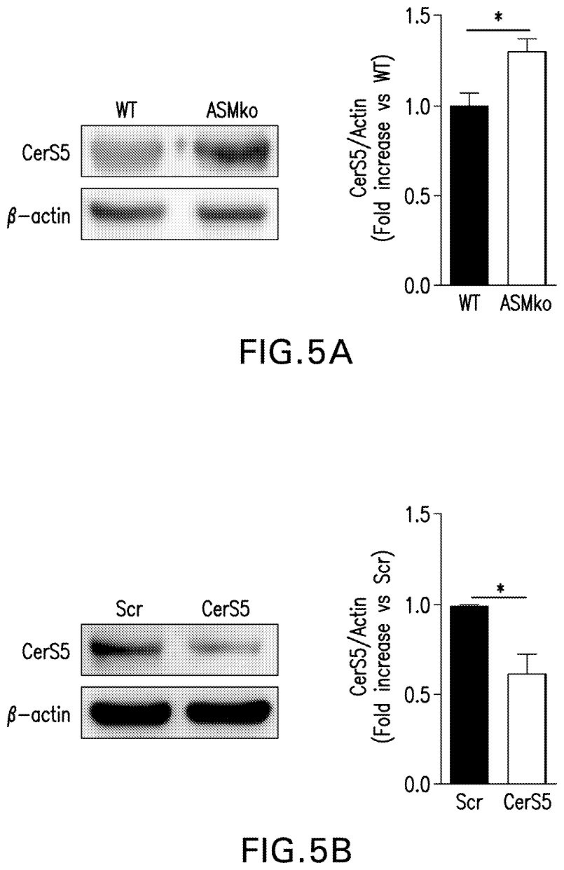

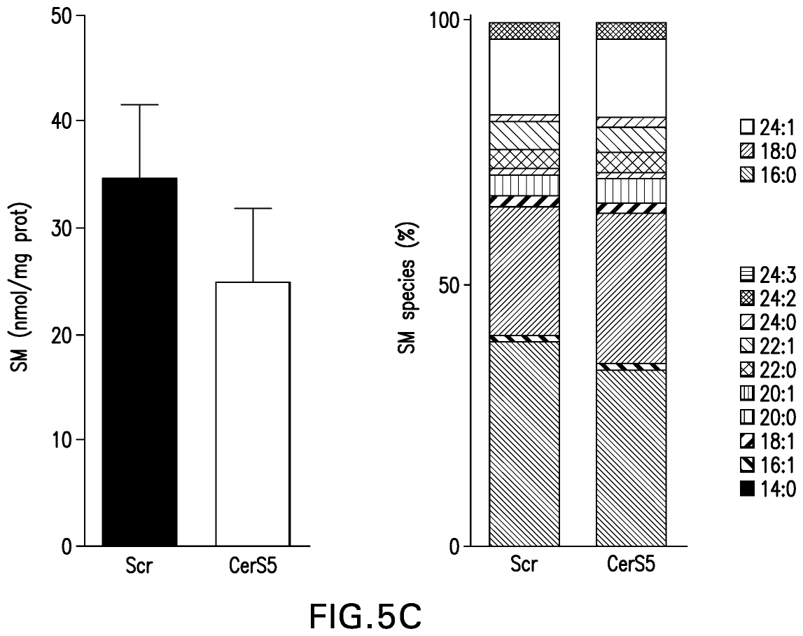

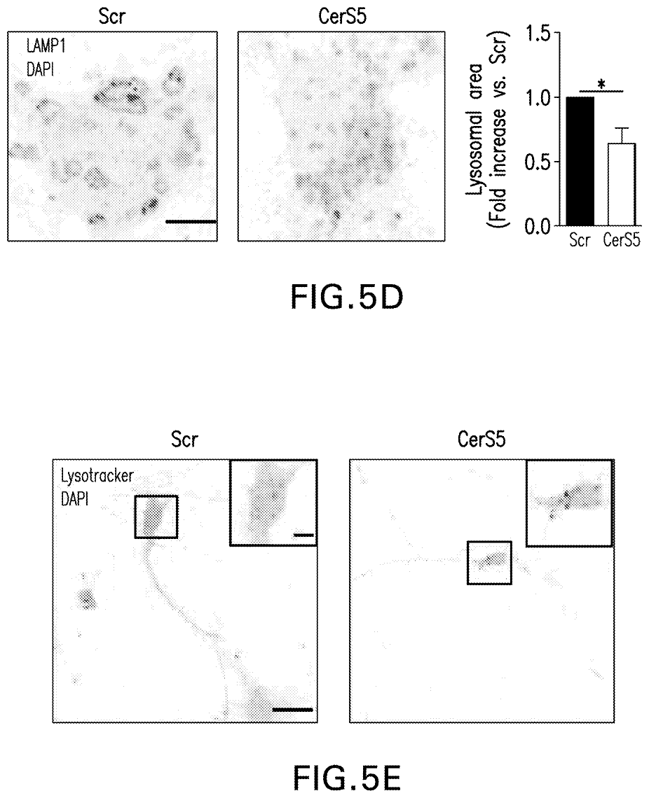

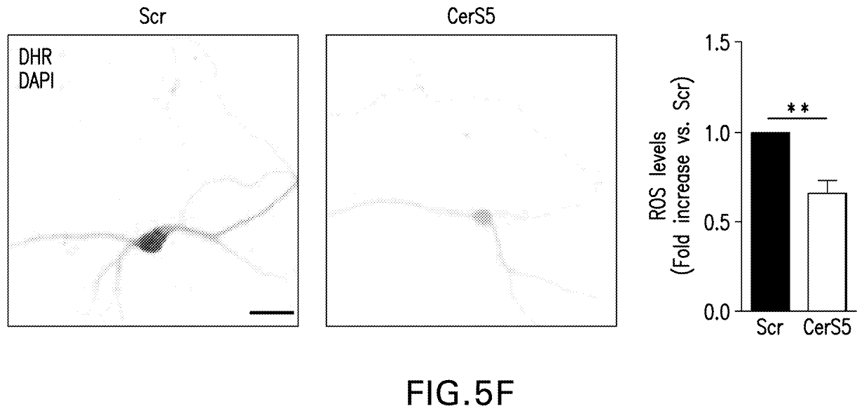

Among the changes in SM metabolic enzymes observed in the ASMko brains was the increase in CerS5, which is involved in the production of SM16:0. It was confirmed by Western blot that, in agreement with its enhanced gene expression (FIG. 4A), the protein levels of CerS5 were 1.3-fold increased in the cerebellum of ASMko mice compared to wt (FIG. 5A). The increased CerS5 gene and protein expression together with the high levels and toxicity of SM16:0 found in the ASMko brain and neurons led us to propose CerS5 inhibition would have beneficial effects. Since compounds that specifically inhibit this enzyme are not currently available its genetic inhibition was evaluated by RNA silencing. To this aim, ASMko cultured cortical neurons were infected with adenovirus containing either shRNA-CerS5 or shRNA-scramble as a control. The efficacy of the treatment was confirmed by the 1.6-fold reduction in the CerS5 levels found by Western Blot in the shRNA-CerS5 compared to the shRNA-scramble infected ASMko cultures (FIG. 5B). A 51% decrease in the levels of dihydroceramide 16:0 was observed, which is a direct metabolite of CerS5 (FIG. 9A). In support of the targeting of the CerS5 silencing on SM16:0, this approach reduced by half the abnormal increase of this SM species in ASMko neurons compared to wt, and raised the relative enrichment of SM18:0 by 5%. It had no effect on SM24:1 (FIG. 5C). Total levels of SM were also not significantly changed by CerS5 silencing (FIG. 5C). Lysosomal parameters were analyzed to determine the effect of the treatment in these organelles. CerS5 silencing significantly reduced lysosomal size (1.6-fold) (FIG. 5D) and lysosomal permeabilization (FIG. 5E) in ASMko cultured neurons. The treatment also diminished oxidative stress (1.5-fold DHR intensity) (FIG. 5F).

### Example 6-Genetic Silencing of CerS5 Ameliorates Pathological Hallmarks in the Brain of ASMko Mice

### Method of AAV9 Infection In Vivo

AAV9 shRNA-scramble or shRNA-CerS5 (2 μl; 5×1012 VG/ml) virus diluted in aCSF was injected at 0.2 μl/min by a glass micropipette into the deep cerebellar nucleus of both hemispheres [anterior-posterior (AP): −5.75 mm; medial-lateral (ML): ±1.8 mm; dorso-ventral (DV): −2.6 mm] of deeply anesthetized animals. Virus injection was performed at 8 weeks of age and experiment was prolonged for another 7 weeks. After surgery, mice were placed in a recovery chamber and were monitored until fully recovered. Then, mice were transferred to their home cages and were monitored daily by trained personnel during the first week after injection and then weekly until the end of the study.

### Results

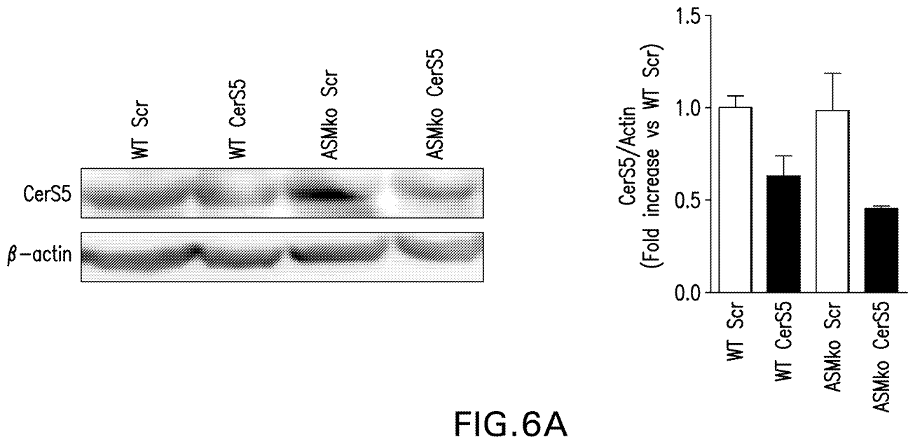

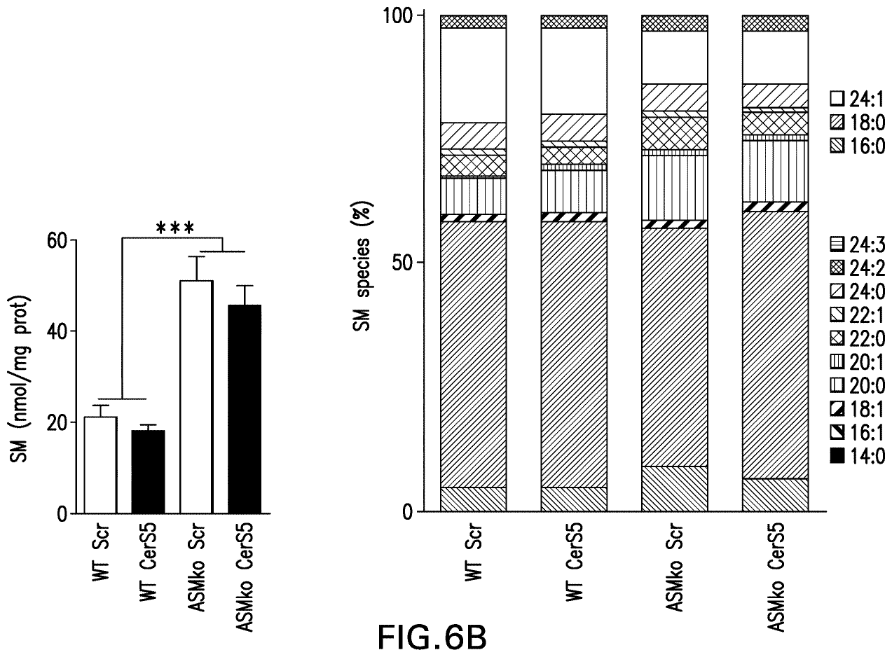

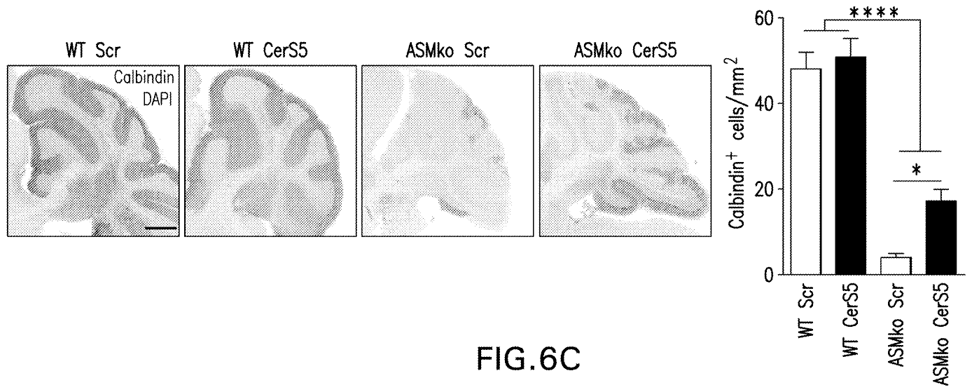

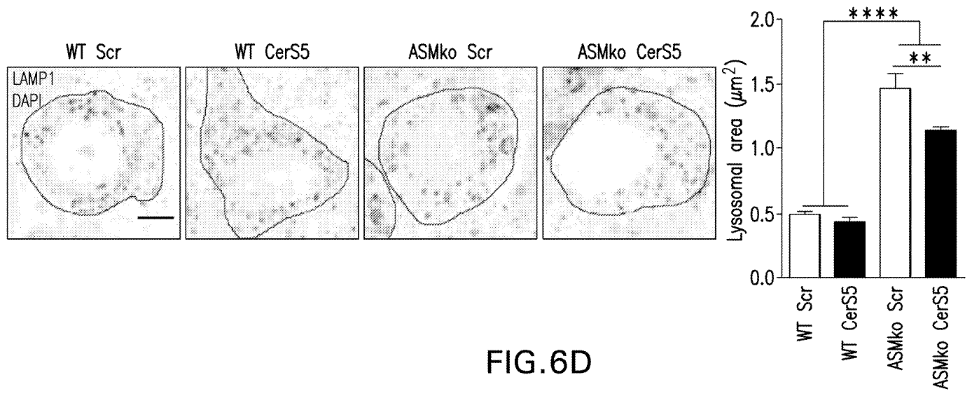

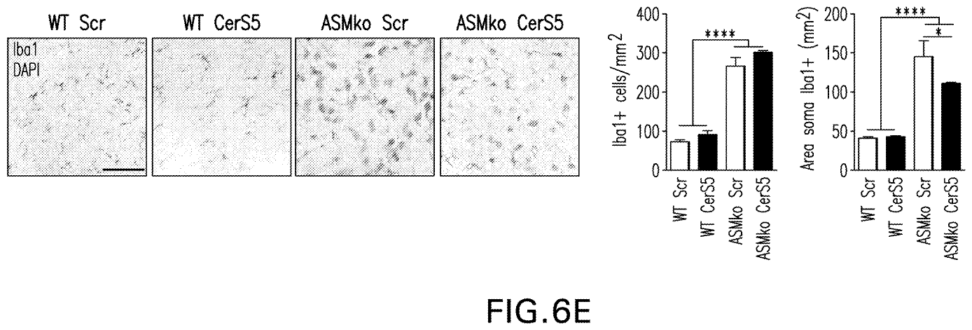

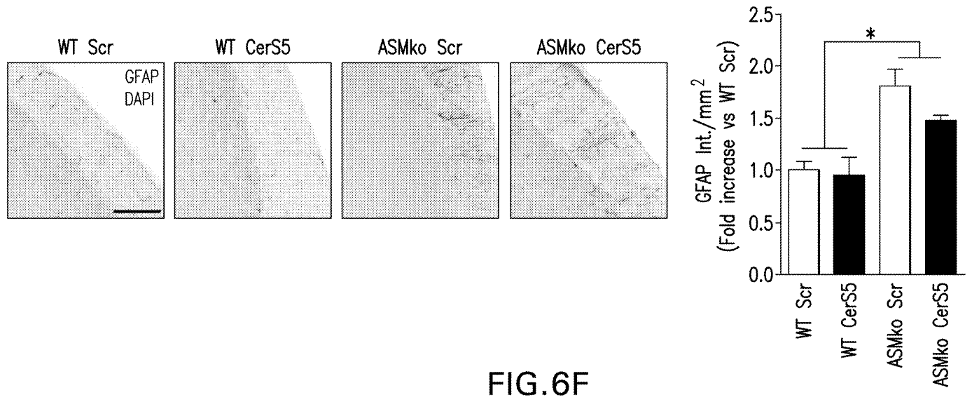

The positive effects of CerS5 genetic silencing in cultured ASMko neurons encouraged assessment of this strategy in the ASMko mice. To this aim, adeno associated serotype 9 viral vectors (AAV9) containing shRNA-scramble or shRNA-CerS5 were used. 5×10¹² VG/ml AAV9-shRNA-scramble or AAV9-shRNA-CerS5 were injected in the earliest and most affected brain area in the disease, the cerebellum, of wt and ASMko mice at 2 months of age. Seven weeks later different cellular and molecular analyses were carried out in the cerebellum. The silencing efficacy was confirmed by Western blot of the levels of CerS5 which were 1.6-fold and 2.2-fold reduced in wt and ASMko mice infected with AAV9-shRNA-CerS5 compared to the AAV9-shRNA-scramble infected (FIG. 6A). Levels of dihidroCer16:0 were also significantly reduced by 43% in the ASMko mice (FIG. 9B). LC/MS analysis of the different SM species in cerebellar extracts showed that CerS5 silencing prevented by half the SM16:0 increase in the ASMko mice (FIG. 6B). Thus, the amount of SM16:0 represented 4% of total SM in the cerebellum of wt mice, raised to 9% in the AAV9-shRNA-scramble injected ASMko mice and was reduced to 6% in the ASMko mice injected with AAV9-shRNA-CerS5 (FIG. 6B). The survival of Purkinje cells, a type of neuron especially vulnerable in the disease, increased by 4.5-fold in the AAV9-shRNA-CerS5 infected ASMko mice as analyzed by immunofluorescence with the specific marker Calbindin (FIG. 6C). The lysosomal size was reduced by 1.3-fold in the Purkinje cells as assessed by immunofluorescence with the marker LAMP1 (FIG. 6D). In addition, inflammation diminished as indicated by the reduction in microglia size detected with the marker iba1 (1.3-fold) (FIG. 6E) and in the intensity of the astrocytic marker GFAP (1.2-fold) (FIG. 6F).

### Example 7—High Levels of SM16:0 in Plasma Correlate with Brain Pathology in ASMko Mice

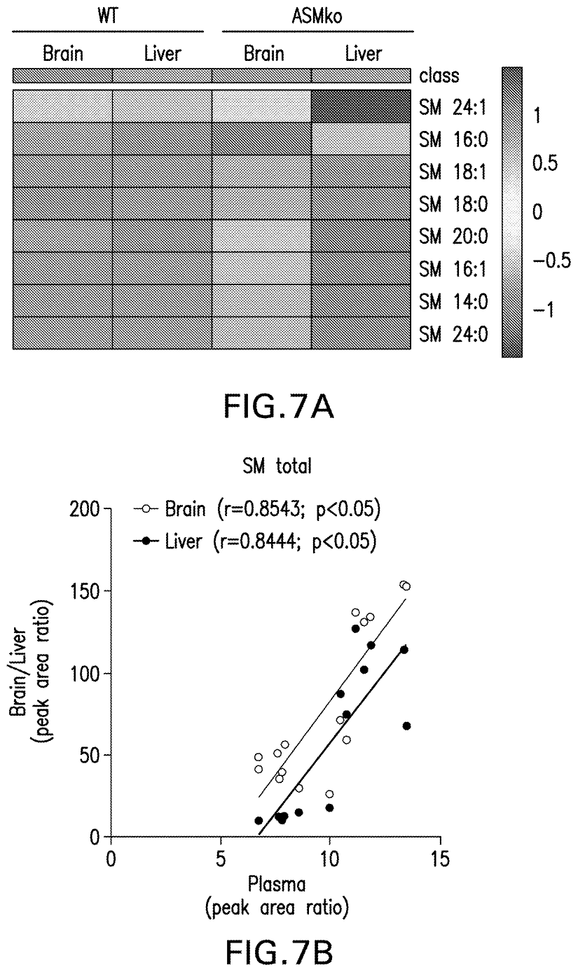

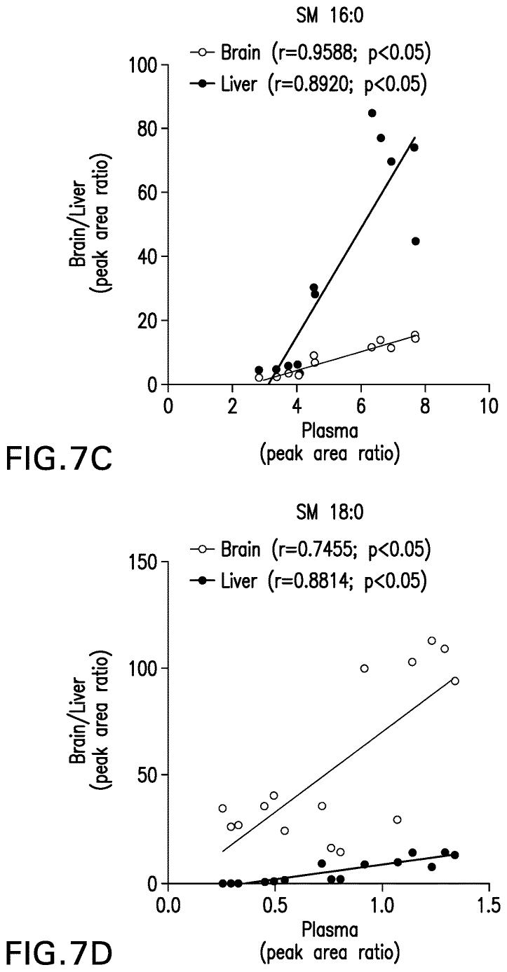

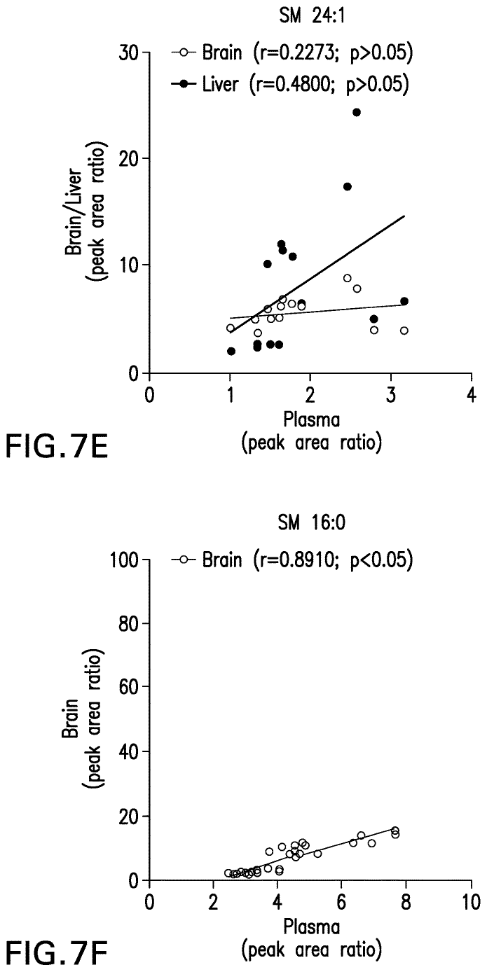

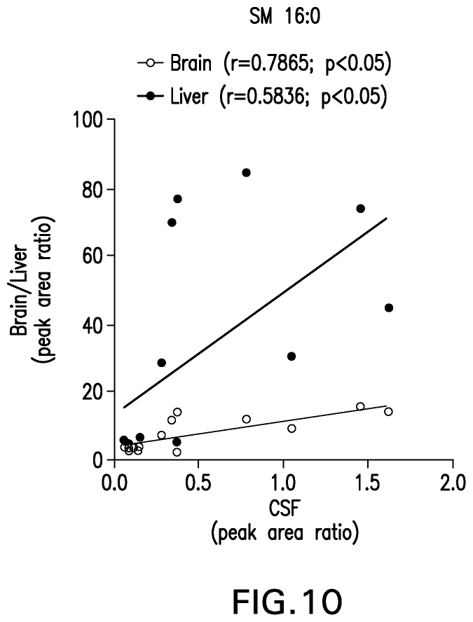

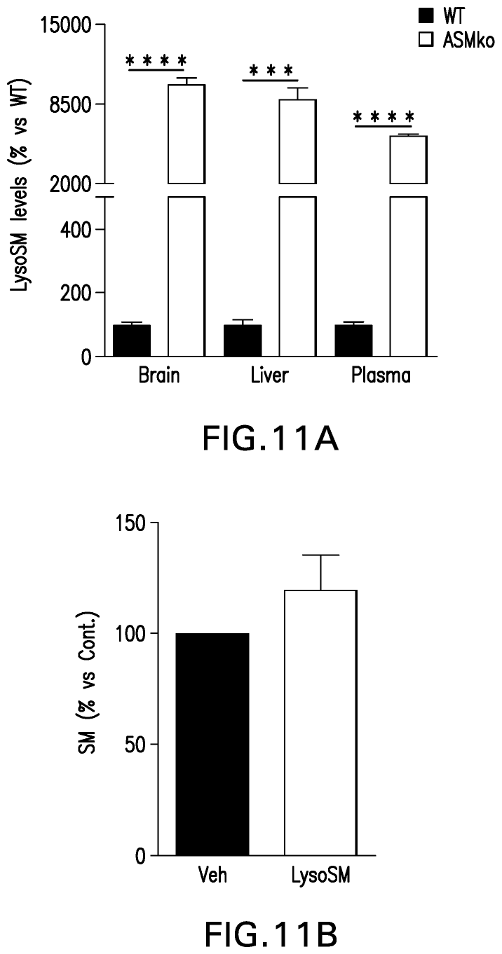

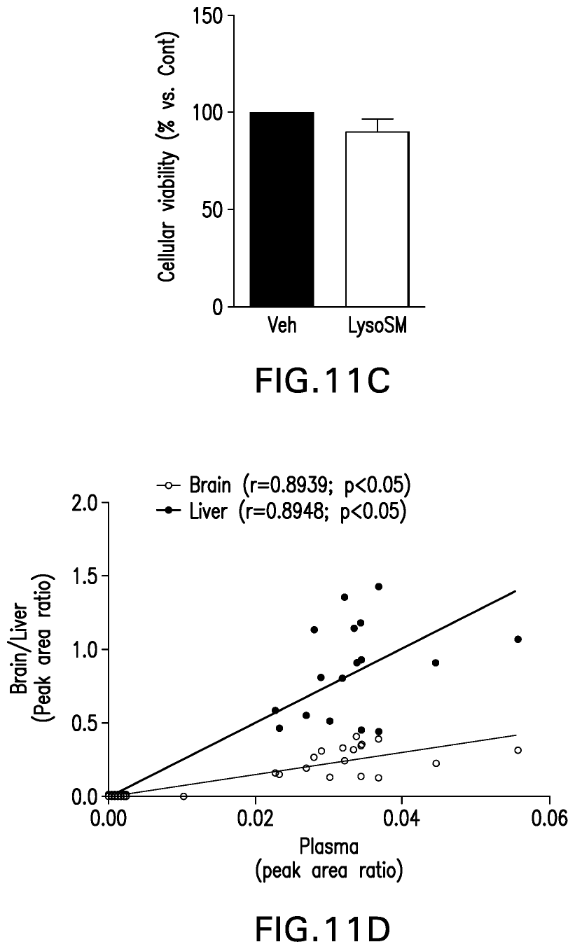

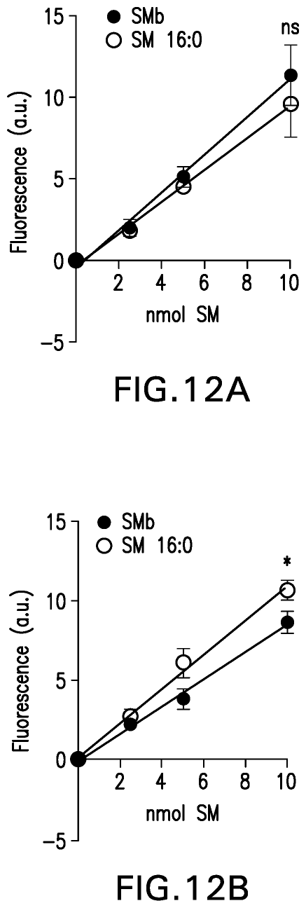

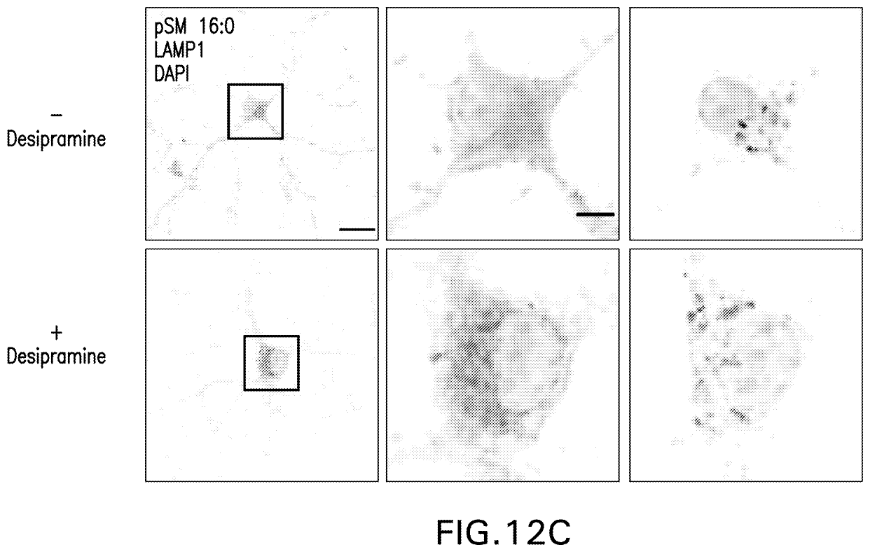

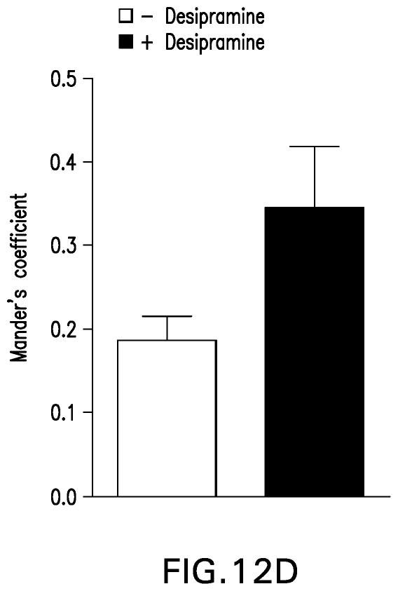

Currently, there is no biomarker specific for brain pathology in ASMD. Given the increase in the ASMko brains of SM16:0 compared to other SM species and its high toxicity for neurons, we postulated that this lipid might be a suitable indicator for neuronal damage in ASMD. To assess this possibility, we first compared the levels of a number of SM species, measured by LC/MS, in brain and liver samples taken from wt and ASMko mice at 4.5 months of age. The LC/MS data were analyzed by the MetaboAnalyst platform, and the relative increments in the SM species levels in ASMko compared to wt values were translated to a color code in a heatmap (FIG. 7A). This analysis unveiled clear differences between liver and brain in the ASMko animals. The increase in all SM species analyzed was higher in liver than in brain except for SM16:0, whose increment was higher in the brain than the liver (FIG. 7A). In contrast, while SM24:1 showed the highest increment in the liver, its levels were unchanged in the ASMko brain compared to the wt situation (FIG. 7A). LC/MS data on the levels of total SM and of SM species in brain and liver also were correlated with those in plasma in 4.5 month-old mice. The correlation coefficient (r) for plasma total SM was similar in brain (r=0.8543) and liver (r=0.8444), ruling out that this measurement can discriminate brain pathology (FIG. 7B). In contrast, SM16:0 levels in plasma showed the highest correlation with the levels in brain (r=0.9588) compared to liver (r=0.8920) (FIG. 7C), while SM18:0 and SM24:1 plasma levels correlated better with liver (r=0.8814 and r=0.4800, respectively) than with brain values (r=0.7455 and r=0.2273, respectively) (FIG. 7D and FIG. 7E). Altogether, these results indicated that SM16:0 in plasma is the SM species that best reflects the changes in the ASMko brain. Levels of SM16:0 in the CSF also correlated better with those in brain (r=0.7865) than in liver (r=0.5836) although with higher variability among samples (FIG. 10). In a recent cross-sectional study in ASMD patients, elevated levels in plasma of the de-acylated form of SM, lyso-sphingomyelin (LysoSM), were found positively associated with clinical severity (Breilyn et al., 2021). The remarkable LysoSM increase was confirmed in the plasma of ASMko mice compared to wt that showed a similar correlation coefficient with both brain and liver levels (r=0.8939 and r=0.8948, respectively) (FIG. 11A). As with total SM levels, this similar correlation does not support the utility of LysoSM to discriminate for brain pathology. Moreover, in contrast to SM 16:0, we did not find deleterious effects upon addition of LysoSM to cultured neurons (FIG. 11A-FIG. 11D).

### Example 8—Biomarker of Brain Pathology in ASMD

### Identification

To identify potential biomarkers of brain pathology in ASMD subjects a study was conducted in ASM knock-out mice. In brief, ASM knock-out mice received an i.p. injection of rhASM at 1 mg/kg every other day for 14 days. Results were compared to previous lipidomic analysis done on WT/ASMko mice at 2, 4, and 6 months.

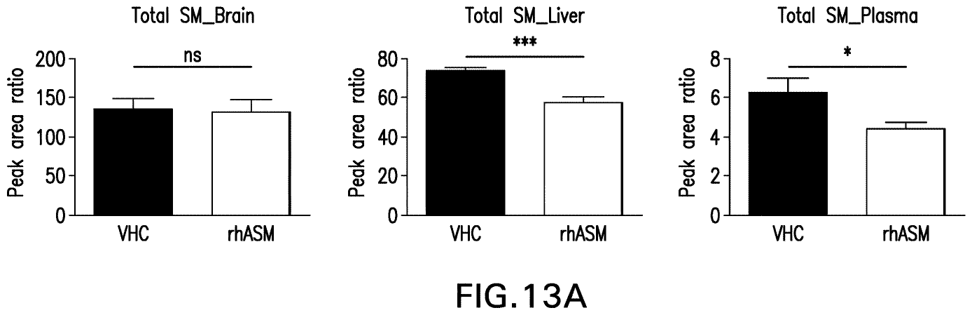

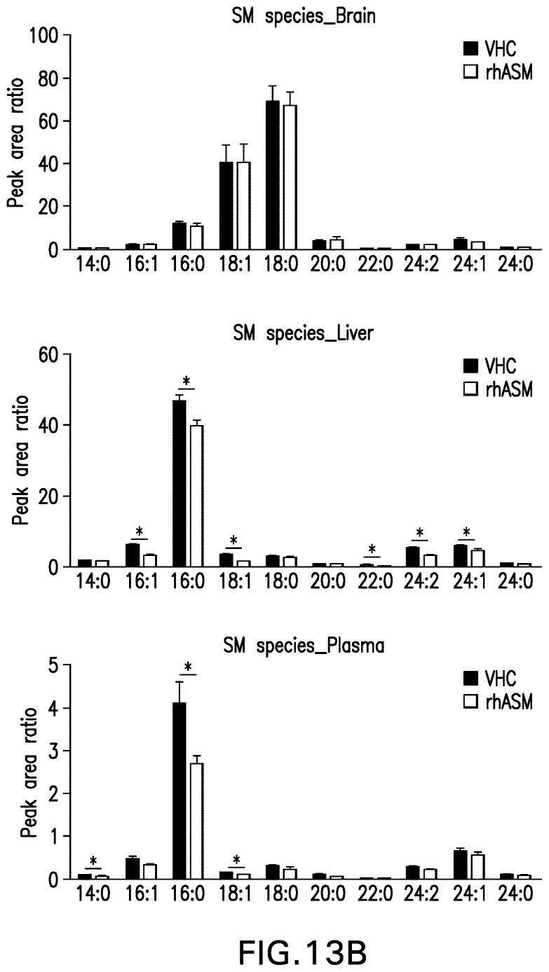

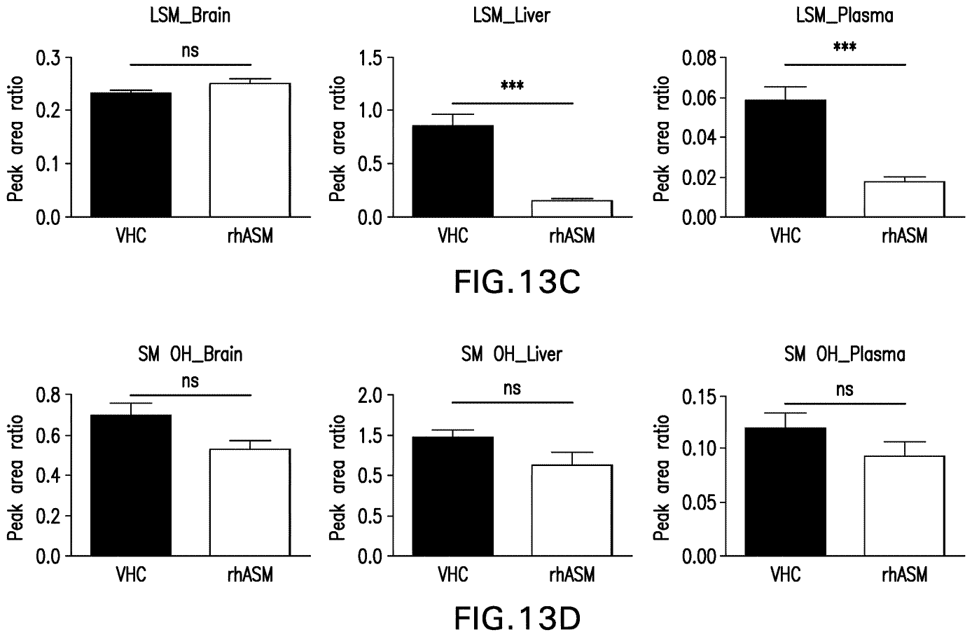

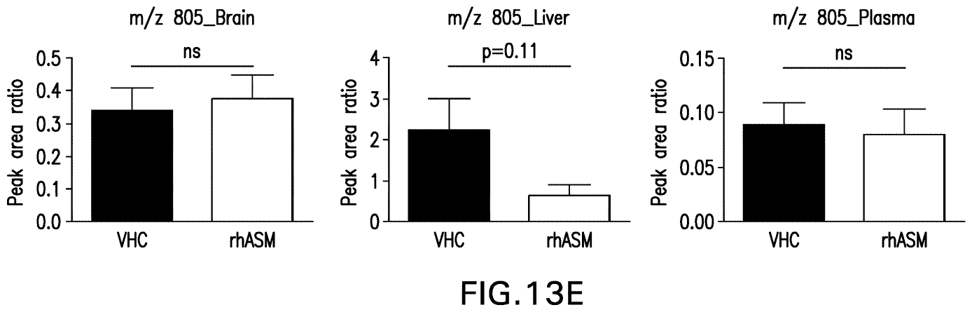

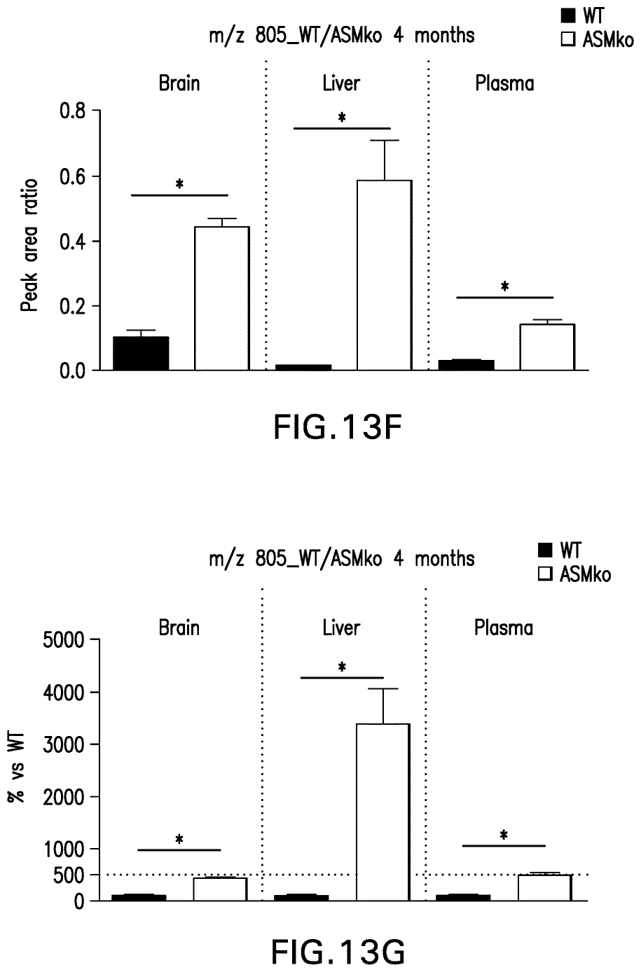

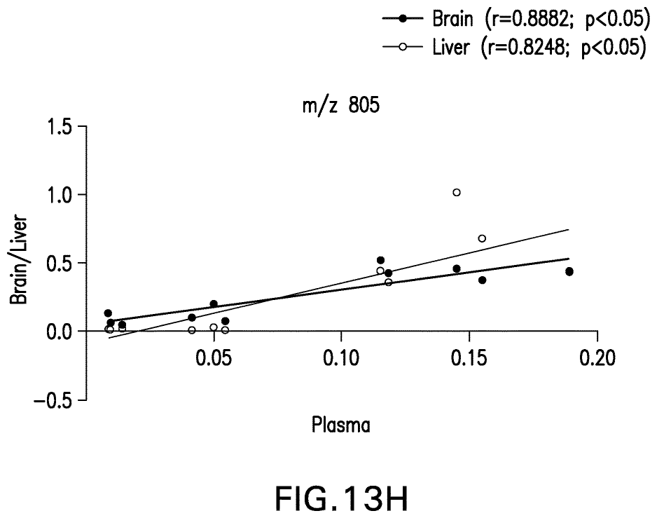

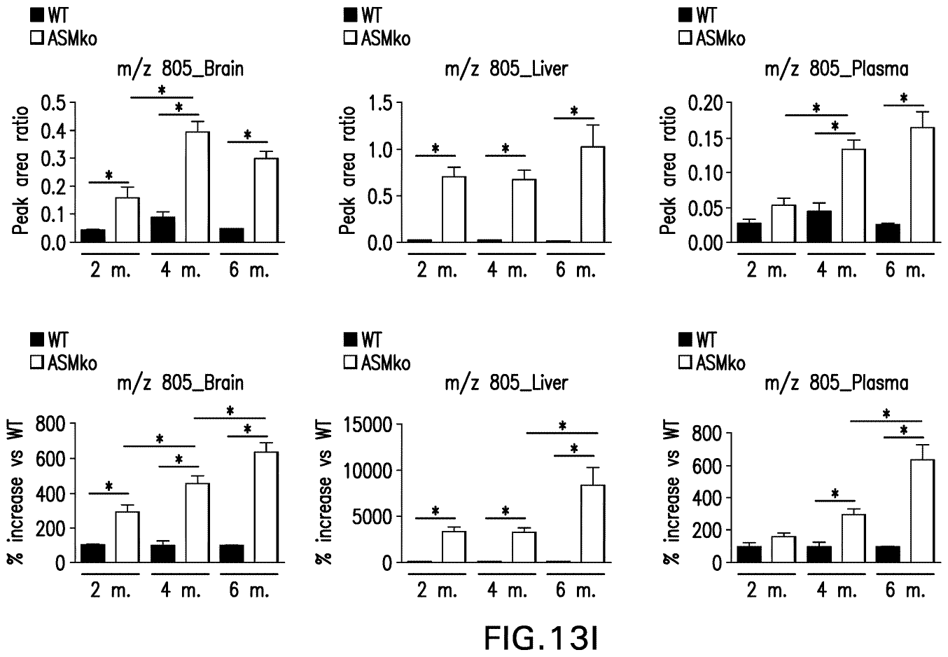

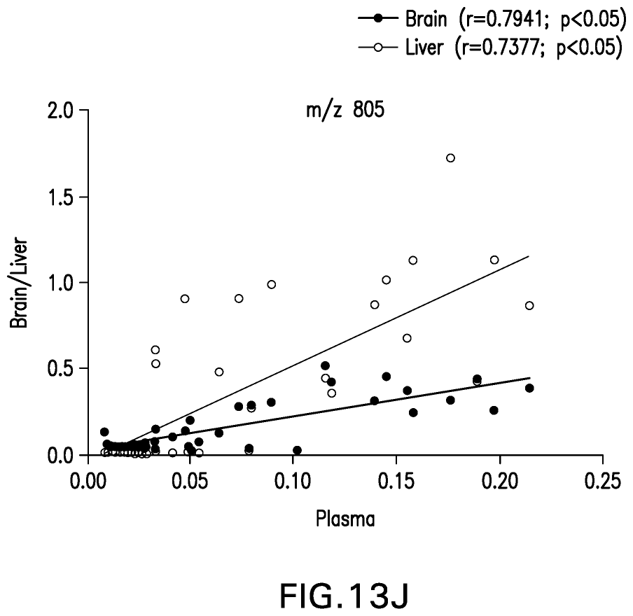

Lipidomic analysis were completed on the brain, liver, and plasma of the treated mice. Analyzed lipids comprised: SM, LSM, SM OH, and m/z 805 (a phosphocholine compound). Total SM levels post i.p. rhASM injection are shown in FIG. 13A and below at Table 3.

TABLE 3
Total SM levels after i.p. rhASM injection

| | % Decrease with rhASM injection |
|---|---|
| Brain | — |
| Liver | 22 |
| Plasma | 31 |

SM species levels after i.p. rhASM injection were evaluated, see FIG. 13B. Results show that SM 16:0 does not change in brain and decreases 15% in liver and 35% in plasma. Additional species, LSM and SM OH levels were also evaluated, see FIG. 13C and Table 4 and FIG. 13D and Table 5. Results show that LSM does not change in the brain and decreases about 83% in the liver and 70% in plasma. SM OH results show a reduction in all samples tested.

TABLE 4
Total LSM levels after i.p. rhASM injection

| | % Decrease with rhASM injection |
|---|---|
| Brain | — |
| Liver | 83 |
| Plasma | 70 |

m/z 805 was also evaluated in the brain, liver, and plasma, see FIG. 13E and Table 5.

TABLE 5
Total m/z 805 levels after i.p. rhASM injection

| | % Decrease with rhASM injection |
|---|---|
| Brain | — |
| Liver | 70 |
| Plasma | — |

Biomarkers were selected according to determination of a decrease in a level thereof in the liver but not in the brain or plasma upon i.p. injection of rhASM. Of the above, m/z 805 showed decreases only in the liver after rhASM injections, indicating it's use as a potential brain pathology marker. Studies of m/z 805 levels in ASMko mice were conducted.

Evaluation of m/z805 in ASMko Mice

ASM loss increase m/z 805 levels in the brain, liver, and plasma, see FIG. 13F in WT/ASMko mice (4 months). FIG. 13G shows the same data as percent change vs WT. Results show that brain and plasma show a similar pattern, about a 500% increase. This pattern was similar to that observed with total SM and SM 16:0. Results show that the brain vs plasma m/z 805 levels have a high correlation coefficient and higher than liver vs. plasma, see FIG. 13H. Further analysis were done using mice of ages 2, 4, and 6 months, see FIG. 13I. In the brain and plasma, there was a relative (% increase vs WT) increase of m/z805 accumulation in the ASMko mice according to age. Indeed, brain vs plasma m/z805 levels have a high correlation coefficient and higher that what was observed in liver vs. plasma. Accordingly m/z805 was selected as a brain pathology marker for ASMD.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method of determining an increased risk of a lysosomal storage disease in a subject, the method comprising determining an increased level of sphingomyelin in the subject as compared to a baseline level of the subject or an otherwise comparable healthy subject.

2. The method of claim 1, wherein the increased level of sphingomyelin is detected in a blood sample of the subject.

3. The method of claim 1, wherein the increased level of sphingomyelin is detected in a sample from the central nervous system of the subject.

4. The method of claim 3, wherein the sample from the central nervous system comprises cerebrospinal fluid.

5. The method of any one of claims 1-4, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.

6. The method of claim 5, wherein the sphingomyelin is sphingomyelin 16:0.

7. The method of any one of claims 1-6, wherein the lysosomal storage disease comprises acid sphingomyelinase deficiency.

8. The method of any one of claims 1-7, wherein the subject is pediatric.

9. The method of claim 8, wherein the subject is an infant.

10. The method of claim 9, wherein the infant is a newborn infant.

11. The method of claim 10, wherein the determining is completed within about 24 hours, 48 hours, or 72 hours of birth.

12. A method of preventing or treating a lysosomal storage disease, the method comprising administering an agent in an amount effective to reduce a level of sphingomyelin in the brain of a subject in need thereof, wherein the agent targets a metabolic enzyme that comprises a ceramide synthase.

13. The method of claim 12, wherein the ceramide synthase is selected from the group consisting of CerS4, CerS5, and CerS6.

14. The method of claim 13, wherein the ceramide synthase is CerS5.

15. The method of any one of claims 12-14, wherein the metabolic enzyme consists of the ceramide synthase.

16. The method of any one of claims 12-15, wherein the agent inhibits the metabolic enzyme, silences an RNA encoding the metabolic enzyme, or genomically disrupts a nucleic acid encoding the metabolic enzyme.

17. The method of claim 16, wherein the agent silences the RNA encoding the metabolic enzyme.

18. The method of claim 17, wherein the agent comprises an adeno-associated virus (AAV) expressing a shRNA that silences the RNA encoding the metabolic enzyme.

19. The method of claim 18, wherein the shRNA comprises a sense sequence, loop sequence, and antisense sequence comprising at least about 80% identity to SEQ ID NO: 23, TCAAGAG, and SEQ ID NO: 24, respectively.

20. The method of claim 19, wherein the shRNA comprises the sense sequence, loop sequence, and antisense sequence of SEQ ID NO: 23, TCAAGAG, and SEQ ID NO: 24, respectively.

21. The method of any one of claims 18-20, wherein the AAV is of a serotype selected from the group consisting of: AAV2, AAV5, AAV6, AAV8, AAV9, a portion thereof, a fusion product thereof, and any combination thereof.

22. The method of claim 21, wherein the AVV is of serotype AAV9.

23. The method of any one of claims 12-22, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.

24. The method of claim 23, wherein the sphingomyelin is sphingomyelin 16:0.

25. The method of any one of claims 12-24, wherein the lysosomal storage disease comprises acid sphingomyelinase deficiency.

26. The method of any one of claims 12-25, wherein the level is reduced by at least about 1-fold, 5-fold, 20-fold, or 50-fold as compared to an otherwise comparable method lacking the administering.

27. The method of any one of claims 12-26, wherein the subject is a pediatric subject.

28. The method of any one of claims 12-26, wherein the subject is an adult.

29. The method of any one of claims 12-28, wherein the subject is concurrently administered recombinant acid sphingomyelinase.

30. The method of any one of claims 12-29, wherein the subject was previously administered recombinant acid sphingomyelinase.

31. A composition comprising:
a) an inhibitor of a ceramide synthase;
b) a short hairpin RNA (shRNA) targeting a ceramide synthase;
c) a guide RNA that targets a nucleic acid sequence encoding a ceramide synthase; or
d) a guide RNA that targets a ribonucleic acid sequence encoding acid sphingomyelinase.

32. The composition of claim 31, wherein the composition comprises the shRNA.

33. The composition of claim 32, wherein the shRNA comprises a sense sequence, loop sequence, and antisense sequence comprising at least about 80% identity to SEQ ID NO: 23, TCAAGAG, and SEQ ID NO: 24, respectively.

34. The composition of claim 33, wherein the shRNA comprises the sense sequence, loop sequence, and antisense sequence of SEQ ID NO: 23, TCAAGAG, and SEQ ID NO: 24, respectively.

35. An AAV particle comprising a sequence encoding a short hairpin RNA (shRNA) targeting a ceramide synthase.

36. The AAV particle of claim 35, wherein the particle is of a serotype selected from the group comprising: AAV2, AAV5, AAV6, AAV8, AAV9, a portion thereof, a fusion product thereof, and any combination thereof.

37. The AAV particle of claim 36, wherein the particle comprises serotype AAV9.

38. The AAV particle of any one of claims 35-37, wherein the shRNA comprises a sense sequence, loop sequence, and antisense sequence comprising at least about 80% identity to SEQ ID NO: 23, TCAAGAG, and SEQ ID NO: 24, respectively.

39. The AAV particle of claim 38, wherein the shRNA comprises the sense sequence, loop sequence, and antisense sequence of SEQ ID NO: 23, TCAAGAG, and SEQ ID NO: 24, respectively.

40. The composition or the AAV particle of any one of claims 31-39, wherein the ceramide synthase is selected from the group consisting of: CerS4, CerS5, and CerS6.

41. The composition or the AAV particle of claim 40, wherein the ceramide synthase is CerS5.

42. A plurality of AAV particles comprising the AAV particle of any one of claims 35-39.

43. The plurality of claim 42, wherein the plurality is in unit dose form.

44. A pharmaceutical composition comprising:
a) the composition of any one of claims 31-34, the AAV particle of any one of claims 35-39, or the plurality of any one of claims 42-43; and
b) a pharmaceutically acceptable excipient, carrier, or diluent.

45. A method of treating or preventing a disease or a condition in a subject in need thereof, the method comprising: administering to the subject the pharmaceutical composition of claim 44 thereby treating or preventing the disease or the condition.

46. The method of claim 45, wherein the administering is intrathecally, intraocularly, intravitreally, retinally, intravenously, intramuscularly, intraventricularly, intracerebrally, intracerebrally, intracerebroventricularly, intraperenchymally, subcutaneously, or a combination thereof.

47. The method of claim 46, wherein the administering is intrathecal.

48. The method of any one of claims 45-47, wherein the disease or condition is a lysosomal storage disease.

49. The method of claim 48, wherein the lysosomal storage disease comprises acid sphingomyelinase deficiency.

50. The method of any one of claims 45-49, wherein after the administering, the subject comprises:
a) at least a 1-fold reduced formation of sphingomyelin as compared to an otherwise comparable subject lacking the administering, as measured by: a brain scan, a blood test, or both; or
b) at least a 1-fold increase in acid sphingomyelinase, as compared to an otherwise comparable subject lacking the administering, as determined a brain scan, a blood test, or both.

51. An AAV particle comprising a sequence encoding a short hairpin RNA (shRNA) targeting a ceramide synthase, wherein the shRNA targets a sequence that is bound by or complementary to at least one of SEQ ID NO: 23 or SEQ ID NO: 25.

52. The AAV particle of claim 51, wherein the AAV particle is of serotype AAV9.

53. The AAV particle of any one of claims 51-52, wherein the ceramide synthase is CerS5.

54. A method of preventing or treating acid sphingomyelinase deficiency comprising administering the AAV particle of any one of claims 51-53 to a subject in need thereof, thereby preventing or treating the acid sphingomyelinase deficiency.

55. The method of claim 54, wherein the administering is effective in reducing an amount of sphingomyelin 16:0 in the subject in need thereof as compared to an otherwise comparable subject lacking the administering.

56. A method of determining whether an agent is neuromodulatory, the method comprising determining a blood plasma concentration of sphingomyelin in a subject treated with an agent, wherein a decrease in the blood plasma concentration as compared to a baseline level is indicative of the efficacy of the compound as a neuromodulatory agent.

57. The method of claim 56, wherein when the decrease is detected, the subject continues treatment with the agent.

58. The method of any one of claims 56-57, wherein the subject has a lysosomal storage disease.

59. The method of claim 58, wherein the lysosomal storage disease comprises acid sphingomyelinase deficiency.

60. The method of any one of claims 56-59, wherein the subject is pediatric.

61. The method of any one of claims 56-60, wherein the sphingomyelin is selected from the group consisting of: sphingomyelin 16:0, sphingomyelin 24:1, sphingomyelin 18:0, sphingomyelin 24:2, sphingomyelin 24:0, sphingomyelin 22:1, sphingomyelin 22:0, sphingomyelin 20:1, sphingomyelin 20:0, sphingomyelin 18:1, sphingomyelin 16:1, sphingomyelin 14:0, and combinations thereof.

62. The method of claim 61, wherein the sphingomyelin is sphingomyelin 16:0.

63. A method of selecting a biomarker, the method comprising:
administering a composition comprising ASM to a subject; and
detecting a level of a lipid in the subject after the administering, wherein when the level of the lipid decreases in a liver of the subject but not a brain or plasma, the lipid is selected.

64. The method of claim 63, wherein the lipid comprises a phosphocholine.

65. The method of claim 64, wherein the phosphocholine comprises m/z805.

66. A composition comprising m/z805.


SEQUENCE LISTING (25 sequences)

SEQ ID NO: 1 - DNA, 20 bp, synthetic construct
  source 1..20: mol_type = other DNA
gcatcagtgccctgtactgt

SEQ ID NO: 2 - DNA, 20 bp, synthetic construct
  source 1..20: mol_type = other DNA
gaccaacttctaccctgggc

SEQ ID NO: 3 - DNA, 21 bp, synthetic construct
  source 1..21: mol_type = other DNA
aataacaatcatcctaagaat

SEQ ID NO: 4 - DNA, 18 bp, synthetic construct
  source 1..18: mol_type = other DNA
tagactcctcatatcctt

SEQ ID NO: 5 - DNA, 20 bp, synthetic construct
  source 1..20: mol_type = other DNA
caactttcgagggtgggtct

SEQ ID NO: 6 - DNA, 20 bp, synthetic construct
  source 1..20: mol_type = other DNA
ttcttcttcccaaagcgcca

SEQ ID NO: 7 - DNA, 18 bp, synthetic construct
  source 1..18: mol_type = other DNA
ttgccttagtcttcttct

SEQ ID NO: 8 - DNA, 18 bp, synthetic construct
  source 1..18: mol_type = other DNA
gatggagtcatacacaga

SEQ ID NO: 9 - DNA, 18 bp, synthetic construct
  source 1..18: mol_type = other DNA
tctacactcctgtgacta

SEQ ID NO: 10 - DNA, 19 bp, synthetic construct
  source 1..19: mol_type = other DNA
agccaataaccttactgaa

SEQ ID NO: 11 - DNA, 18 bp, synthetic construct
  source 1..18: mol_type = other DNA
tactgctctgacgacttg

SEQ ID NO: 12 - DNA, 18 bp, synthetic construct
  source 1..18: mol_type = other DNA
gctgttgctgctattctc

SEQ ID NO: 13 - DNA, 18 bp, synthetic construct
  source 1..18: mol_type = other DNA
cgttggaatcttctgtat

SEQ ID NO: 14 - DNA, 18 bp, synthetic construct
  source 1..18: mol_type = other DNA
cttgtggtgatgtagtag

SEQ ID NO: 15 - DNA, 19 bp, synthetic construct
  source 1..19: mol_type = other DNA
aaaggccacctgtctgtcc

SEQ ID NO: 16 - DNA, 20 bp, synthetic construct
  source 1..20: mol_type = other DNA
ggcctgaccaatgctctctt

SEQ ID NO: 17 - DNA, 20 bp, synthetic construct
  source 1..20: mol_type = other DNA
tgggacatcccctacctgag

SEQ ID NO: 18 - DNA, 21 bp, synthetic construct
  source 1..21: mol_type = other DNA
taggtgagcgatagcctttgc

SEQ ID NO: 19 - DNA, 21 bp, synthetic construct
  source 1..21: mol_type = other DNA
acacgaccccctttcctaata

SEQ ID NO: 20 - DNA, 20 bp, synthetic construct
  source 1..20: mol_type = other DNA
ggcgcttctcataggtggtg

SEQ ID NO: 21 - DNA, 21 bp, synthetic construct
  source 1..21: mol_type = other DNA
cacactagcctcttgaagcga

SEQ ID NO: 22 - DNA, 22 bp, synthetic construct
  source 1..22: mol_type = other DNA
tgtagaacctccaacttggcat

SEQ ID NO: 23 - DNA, 21 bp, synthetic construct
  source 1..21: mol_type = other DNA
gcatgtggagattcacttatt

SEQ ID NO: 24 - skipped

SEQ ID NO: 25 - DNA, 21 bp, synthetic construct
  source 1..21: mol_type = other DNA
aataagtgaatctccacatgc